(19) United States
(12) Patent Application Publication
(10) Pub. No.: US 2024/0197660 A1
(43) Pub. Date: Jun. 20, 2024

(54) COMPOSITIONS OF MICRONIZED SOLABEGRON AND METHODS OF USE

(71) Applicant: B3AR THERAPEUTICS, INC., PALO ALTO, CA (US)

(72) Inventors: Raymond E. STEVENS, Jr., West Chester, PA (US); Dale BENNYHOFF, Devon, PA (US)

(73) Assignee: B3AR THERAPEUTICS, INC., PALO ALTO, CA (US)

(21) Appl. No.: 18/550,790

(22) PCT Filed: Mar. 31, 2022
(86) PCT No.: PCT/US2022/071462

§ 371 Date: Sep. 15, 2023

Related U.S. Application Data
(60) Provisional application No. 63/168,817, filed on Mar. 31, 2021.

(51) Int. Cl. A61K 31/196; A61K 9/20; A61K 31/137; A61K 31/438

(52) U.S. Cl. CPC A61K 31/196; A61K 9/2009; A61K 9/2027; A61K 9/2054; A61K 9/2077; A61K 31/137; A61K 31/438

(57) ABSTRACT

Disclosed herein are compositions and methods for treating overactive bladder. In some embodiments, the composition comprises an effective amount of micronized solabegron or a pharmaceutically acceptable salt or a derivative thereof. In some embodiments the micronized solabegron particles have a particle size of about 0.1 micron to 30 microns. In some embodiments, the administration of the pharmaceutical composition is twice daily.

### CROSS REFERENCE TO RELATED APPLICATION

The present application is a U.S. national stage filing under 35 U.S.C. § 371 of International Application No. PCT/US2022/071462 filed Mar. 31, 2022, entitled “COMPOSITIONS OF MICRONIZED SOLABEGRON AND METHODS OF USE”, which claims benefit of priority under 35 U.S.C. § 119(e) to U.S. Provisional Application Ser. No. 63/168,817 entitled “COMPOSITIONS OF MICRONIZED SOLABEGRON AND METHODS OF USE”, filed Mar. 31, 2021, the contents of which is hereby incorporated by reference in its entirety.

### SUMMARY

Disclosed herein are solid, orally administrable pharmaceutical compositions and methods for treating overactive bladder comprising administering such pharmaceutical compositions to a subject in need thereof.

In some embodiments, the pharmaceutical composition comprises about 75 mg to about 125 mg of micronized solabegron or a pharmaceutically acceptable salt or a derivative thereof, and a pharmaceutically acceptable excipient, wherein at least 90% of the solabegron particles have a particle size of about 0.1 micron to 30 microns. In some embodiments the excipient is selected from the group consisting of a filler, a disintegrant, a binder, a wetting agent, a lubricant, and a glidant, or combinations thereof. In some embodiments the excipient is selected from the group consisting of mannitol, poloxamer 188, methyl cellulose, croscarmellose sodium, magnesium stearate, colloidal silicon dioxide, and polyvinyl alcohol, or combinations thereof. In some embodiments the excipient is selected from the group consisting of sucrose, wheat starch, microcrystalline cellulose, talc, lactose monohydrate, calcium carbonate, titanium dioxide, stearic acid, croscarmellose sodium, povidone, polyethylene glycol 8000, colloidal silicon dioxide, ferric oxide, carboxymethylcellulose sodium, white wax, magnesium stearate, and carnauba wax or combinations thereof. In some embodiments, the excipient is selected from the group consisting of colloidal anhydrous silica, calcium hydrogen phosphate dihydrate, cellulose microcrystalline, hypromellose, magnesium stearate, sodium starch glycolate (pH 3.0 to 5.0), stearic acid, and titanium dioxide or combinations thereof. In some embodiments, the pharmaceutical composition further comprises one or more additional therapeutic agents selected from the group consisting of antimuscarinic agents, alpha adrenoceptor blockers, botulinum toxin, purinergics, cannabinoids, transient receptor potential (TRP) protein inhibitors, prostaglandins, percutaneous tibial nerve stimulators, sacral nerve stimulators, 5-alpha reductase inhibitors, phosphodiesterase-5 inhibitors, and combination thereof. In some embodiments, the pharmaceutical composition further comprises one or more additional therapeutic agents selected from trospium or tolterodine. In some embodiments, the pharmaceutical composition is a tablet, a capsule, a granule, or a powder. In some embodiments, the pharmaceutical composition is an immediate release composition, a modified release composition, or a combination thereof.

Embodiments herein describe a method of treating overactive bladder or one or more symptoms thereof, in a subject in need thereof, comprising orally administering to the subject a pharmaceutical composition comprising a therapeutically effective amount of about 75 mg to about 125 mg of micronized solabegron or a pharmaceutically acceptable salt or a derivative thereof, and a pharmaceutically acceptable excipient, wherein at least 90% of the solabegron particles have a particle size of about 0.1 micron to 30 microns. In some embodiments, the pharmaceutical composition is an immediate release composition. In some embodiments, the administration of the pharmaceutical composition is twice daily. In some embodiments, the one or more symptoms of overactive bladder are selected from increased frequency of urinary urgency, nocturia, increase in urinary micturition frequency, and urinary incontinence, and combination thereof. In some embodiments the method comprises administering to the subject one or more additional therapeutic agents selected from the group consisting of antimuscarinic agents, alpha adrenoceptor blockers, botulinum toxin, purinergics, cannabinoids, transient receptor potential (TRP) protein inhibitors, prostaglandins, percutaneous tibial nerve stimulators, sacral nerve stimulators, 5-alpha reductase inhibitors, phosphodiesterase-5 inhibitors, beta-3 adrenoreceptor agonists, and combination thereof. In some embodiments the additional therapeutic agent is an anti-muscarinic agent. In some embodiments the anti-muscarinic agent is trospium or tolterodine.

Embodiments of the present invention are also directed to methods of treating irritable bowel syndrome and similar gastrointestinal disorders in a subject in need thereof comprising orally administering to the subject a pharmaceutical composition comprising a therapeutically effective amount of about 50 mg to about 125 mg of micronized solabegron or a pharmaceutically acceptable salt or a derivative thereof, and a pharmaceutically acceptable excipient, wherein at least 90% of the solabegron particles have a particle size of about 0.1 micron to 30 microns. In some embodiments, the pharmaceutical composition is an immediate release composition. In some embodiments, the administration of the pharmaceutical composition is twice daily.

Embodiments of the present invention are also directed to methods of treating inflammatory bowel disease in a subject in need thereof comprising orally administering to the subject a pharmaceutical composition comprising a therapeutically effective amount of about 50 mg to about 125 mg of micronized solabegron or a pharmaceutically acceptable salt or a derivative thereof, and a pharmaceutically acceptable excipient, wherein at least 90% of the solabegron particles have a particle size of about 0.1 micron to 30 microns. In some embodiments, the pharmaceutical composition is an immediate release composition. In some embodiments, the administration of the pharmaceutical composition is twice daily.

Embodiments of the present invention are also directed to methods of treating overactive bladder or one or more symptoms thereof, in a subject in need thereof, comprising orally administering to the subject a pharmaceutical composition comprising a therapeutically effective amount of micronized solabegron or a pharmaceutically acceptable salt or a derivative thereof, wherein the pharmaceutical composition achieves a target AUC₄₈ of about 17,000 ng·hr/mL to about 23,000 ng·hr/mL. In some embodiments, the therapeutically effective amount is about 100 mg of micronized solabegron and is administered twice daily. In some embodiments, the therapeutically effective amount is about 100 mg of micronized solabegron and is administered once daily.

Embodiments of the present invention are also directed to methods of treating overactive bladder or one or more symptoms thereof, in a subject in need thereof, comprising orally administering to the subject a pharmaceutical composition comprising a therapeutically effective amount of micronized solabegron or a pharmaceutically acceptable salt or a derivative thereof, wherein the pharmaceutical composition achieves a target AUC₄₈ of about 14,000 ng·hr/mL to about 29,000 ng·hr/mL. In some embodiments, the therapeutically effective amount is about 100 mg of micronized solabegron and is administered twice daily. In some embodiments, the therapeutically effective is about 100 mg of micronized solabegron and is administered once daily.

### BRIEF DESCRIPTION OF THE FIGURES

FIG. 1 shows a dissolution profile of a non-micronized 75 mg solabegron tablet composition.

FIG. 2 shows a dissolution profile of a non-micronized 100 mg solabegron tablet composition.

FIG. 3 shows a profile of a non-micronized 125 mg solabegron tablet composition.

FIG. 4 shows a dissolution profile of a non-micronized 175 mg solabegron tablet composition.

FIGS. 5A and 5B show an exemplary dissolution profile of a micronized 125 mg solabegron tablet composition (A) and the corresponding values at different time points (B).

FIGS. 6A and 6B show an exemplary dissolution profile of a micronized 175 mg solabegron tablet composition (A) and the corresponding values at different time points (B).

FIG. 7 shows a comparison of dissolution profiles of non-micronized and micronized 125 mg solabegron tablets.

FIG. 8 shows a comparison of dissolution profiles of non-micronized and micronized 175 mg solabegron tablets.

FIGS. 9A and 9B show efficacy data of micronized solabegron used in Phase 2b clinical study of Example 5.

FIG. 10 shows a comparison of clinical efficacy of non-micronized solabegron 125 mg and micronized solabegron 125 mg. Clinical meaningful improvement is 15%-20% improvement vs. current 03 treatment Myrbetriq.

### DETAILED DESCRIPTION

As used herein, the term “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation.

As used herein the term “agonist” refers to a compound, the presence of which results in a biological activity of a receptor that is the same as the biological activity resulting from the presence of a naturally occurring ligand for the receptor.

The terms “administer,” “administering” or “administration” as used herein refer to either directly administering a compound (e.g., solabegron) or pharmaceutically acceptable salt of the compound (e.g., solabegron) or a composition comprising solabegron or a pharmaceutically acceptable salt thereof to a subject.

As used herein the term “b.i.d.” mean twice a day (from the Latin bis in die).

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention. In embodiments or claims where the term comprising is used as the transition phrase, such embodiments can also be envisioned with replacement of the term “comprising” with the terms “consisting of” or “consisting essentially of.”

As used herein, the term “derivative” refers to pharmaceutically acceptable solvates, pharmaceutically acceptable salts solvated with pharmaceutically acceptable solvents thereof, and metabolites.

As used herein, the term “effective amount” refers to an amount that results in measurable inhibition of at least one symptom or parameter of a specific disorder or pathological process. As used herein the term “therapeutically effective amount” of compositions of the application is an amount, which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect or physician observes a change).

As used herein the term “immediate release” refers to pharmaceutical compositions that release the active ingredient within a short period of time.

As used herein the term “modified release” refers to pharmaceutical compositions that does not otherwise release the active ingredient immediately, for example it may release the active ingredient at a sustained or controlled rate over an extended period of time or may release the active ingredient after a lag time after administration or may be used optionally in combination with an immediate release composition. Modified release includes extended release, sustained release and delayed release. The term “extended release” or “sustained release” as used herein is a dosage form that makes a drug available over an extended period of time after administration. The term “delayed release” as used herein is a dosage form that releases a drug at a time other than immediately upon administration.

As used herein the phrase “overactive bladder” or “OAB” refers to a group of medical symptoms comprising urinary urgency, frequent urination, nocturia, urinating unintentionally, increase in urinary micturition frequency, and urinary incontinence. Subjects with OAB may have one or more of these symptoms.

The phrase “pharmaceutically acceptable” refers to molecular entities and compositions that are generally regarded as safe and nontoxic. In particular, pharmaceutically acceptable carriers, diluents or other excipients used in the pharmaceutical compositions of this application are physiologically tolerable, compatible with other ingredients, and do not typically produce an allergic or similar untoward reaction (e.g., gastric upset, dizziness and the like) when administered to a subject. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

The phrase “pharmaceutically acceptable salt(s)”, as used herein, includes those salts of compounds of the application that are safe and effective for use in mammals and that possess the desired biological activity. Pharmaceutically acceptable salts include salts of acidic or basic groups present in compounds of the application or in compounds identified pursuant to the methods of the application. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the application can form pharmaceutically acceptable salts with various amino acids. Suitable base salts include, but are not limited to, aluminum, calcium, lithium, magnesium, potassium, sodium, zinc, iron and diethanolamine salts. Pharmaceutically acceptable base addition salts are also formed with amines, such as organic amines. Examples of suitable amines are N,N′-dibenzylethylenediamine, chloroprocaine, choline, diethanolamine, dicyclohexylamine, ethylenediamine, N-methylglucamine, and procaine.

The terms “subject,” “individual” or “patient” are used interchangeably and as used herein are intended to include human and non-human animals. Non-human animals include all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include humans in need of treatment. The methods are particularly suitable for treating humans having a disease or disorder described herein.

As used herein, the term “therapeutic” means an agent utilized to treat, combat, ameliorate, protect against or improve an unwanted condition or disease of a subject.

As used herein the terms “treat”, “treated”, or “treating” refer to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects.

The “weight percent” disclosed herein may be weight-to-weight percent or weight-to-volume percent, depending upon the composition.

Overactive bladder (OAB) is a condition characterized by sudden, involuntary contraction of the detrusor muscle of the urinary bladder. This results in a sudden, compelling need to urinate that is difficult to suppress (urinary urgency), even though the bladder may only contain a small amount of urine. The key symptom is the sudden urge to void (urgency) with or without urgency urinary incontinence, often associated with urinary frequency (voiding 8 or more times per day) and nocturia (awakening one or more times at night to void). Overactive bladder coupled with urinary leakage (inability to suppress the urge to void) is also referred to as urgency urinary incontinence.

As used herein, the term “urinary urgency” is considered the hallmark symptom of OAB, and is the sudden, compelling desire to pass urine that is difficult to defer.

As used herein, the term “urinary frequency” or “frequency of micturitions” refers to the number of times a subject voids and is considered abnormal if the person urinates more than eight times in a day. This frequency is usually monitored by having the person record urination episodes in a voiding diary. The number of episodes varies depending on sleep, fluid intake, medications, and up to seven is considered normal if consistent with the other factors.

The term “nocturia,” as used herein, is a symptom where the person complains of interrupted sleep because of an urge to void and, similar to the urinary frequency component, is affected by similar lifestyle and medical factors. Individual waking events are not considered abnormal.

As used herein, the term “urgency urinary incontinence” is a form of urinary incontinence characterized by the involuntary loss of urine occurring for no apparent reason while feeling urinary urgency as discussed above. Urgency urinary incontinence can be measured with pad tests, and these are often used for research purposes. The goal in treating urgency urinary incontinence is to reduce the number of leakage episodes.

As used herein, the term “voided volume” is used as a measure of bladder capacity. Anatomically, functional bladder capacity increases with age from childhood [(years of age+2)×30 mL] to adulthood (300-600 mL). A goal when treating OAB is to increase the bladder capacity or voided volume. An increase in voided volume is a good indicator of the efficacy of a therapy. For example, a goal of OAB treatment is to decrease micturition frequency. This is one of the recognized endpoints for treatment of OAB. Accordingly, if voided volume increases, and intake remains the same, then the number of micturitions (i.e., micturition frequency) will decrease.

Normally, the kidneys produce urine, which drains into the bladder. During urination, urine passes from the bladder through the urethra. As the bladder fills, afferent nerve signals sent to the brain trigger the need to urinate, and the nerve signals coordinate the relaxation of the pelvic floor muscles and the muscles of the urethra (urinary sphincter muscles). The muscles of the bladder contract, pushing the urine out (micturition). Overactive bladder occurs because the muscles of the bladder start to contract involuntarily even when the volume of urine in the bladder is low. This involuntary contraction creates the sensation of an urgent need to urinate. Approximately 300 ml of urine in the bladder can signal the nerves to trigger muscles of the bladder to coordinate urination. Voluntary control of the sphincter muscles at the opening of the bladder can hold the urine in the bladder for longer. Up to 600 ml of urine can be contained in a normal adult bladder. For those with OAB, the bladder capacity is typically low (<200 ml).

OAB treatment goals include decreasing the frequency of urinary urgency, decreasing nocturia, decreasing urinary micturition frequency, decreasing urinary incontinence, increasing voided volume, decreasing post-void residual volume, improving patient reported outcomes, and combination thereof.

Disclosed herein are pharmaceutical compositions of micronized solabegron or a pharmaceutically acceptable salt or a derivative thereof, and methods for treating overactive bladder.

Solabegron (3′-[(2-{[(2R)-2-(3-chlorophenyl)-2-hydroxyethyl]amino}ethyl) amino]biphenyl-3-carboxylic acid) is a beta-3 adrenoceptor agonist, with the following structure:

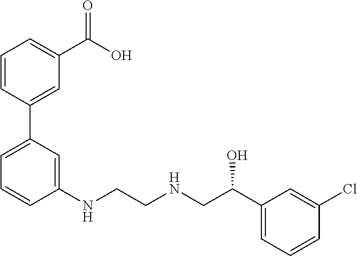 [SMILES: O=C(O)c1cccc(-c2cccc(NCCNC[C@H](O)c3cccc(Cl)c3)c2)c1]

It is further described in U.S. Pat. Nos. 6,251,925, 8,642,661, 9,907,767, and PCT Application No. US2015/38583.

In some embodiments, a pharmaceutical composition comprises a therapeutically effective amount of micronized solabegron or a pharmaceutically acceptable salt thereof. In embodiments, micronized solabegron is amorphous, zwitterion or the free base. In embodiments, a pharmaceutically acceptable salt thereof may include, but is not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)), various amino acids, aluminum, calcium, lithium, magnesium, potassium, sodium, zinc, iron, diethanolamine, amines, such as organic amines, N,N′-dibenzylethylenediamine, chloroprocaine, choline, diethanolamine, dicyclohexylamine, ethylenediamine, N-methylglucamine, and procaine. Solabegron may exist in any physical form known to one of skill in the art such as, for example, nanoparticles, crystalline solids, amorphous solids, polymorphs, ionic solids such as, for example, cations, anions and zwitterions, pharmaceutically acceptable salts, hydrates, solvates, stereoisomers, and suspensions. Crystalline solids have regular ordered arrays of components held together by uniform intermolecular forces, whereas the components of amorphous solids are not arranged in regular arrays. Hydrates are substances that incorporate at least one water molecule into their crystalline matrix. Solvates are substances that incorporate at least one solvent molecule into their crystalline matrix. Polymorphs exhibit different crystalline structures for molecules that have the same molecular formula and sequence of bonded atoms. Stereoisomers are isomeric molecules that have the same molecular formula and sequence of bonded atoms (constitution), but that differ only in the three-dimensional orientations of their atoms in space. In some embodiments, solabegron is the amorphous solid form of solabegron. In some embodiments, solabegron is solabegron hydrochloride. In some embodiments, the solabegron is the zwitterion form of solabegron.

Without wishing to be bound by theory, it was observed that decreasing the particle size of solabegron active pharmaceutical ingredient (API) to 30 microns, in a tablet formulation, resulted in better pharmacokinetics, such as increased AUC (24 hrs.) when compared to a tablet having a solabegron particle size of more than 30 microns. This observation was unexpected and surprising and demonstrates the impact of decreasing the particle size (micronization) of the solabegron in the pharmaceutical composition on the pharmacokinetics.

### Micronized Solabegron Compositions

In some embodiments, the pharmaceutical composition comprises micronized solabegron or a pharmaceutically acceptable salt or a derivative thereof. The micronized solabegron particles may have an average particle size about 0.1 micron to about 30 microns, about 0.1 micron to about 25 microns, about 0.1 micron to about 20 microns, about 0.1 micron to about 10 microns, about 0.1 micron to about 8 microns, about 0.1 micron to about 6 microns, about 0.1 micron to about 4 microns, about 0.1 micron to about 2 microns, about 0.1 micron to about 1 micron, about 1 micron to about 10 microns, about 1 micron to about 9 microns, about 1 micron to about 8 microns, about 1 micron to about 7 microns, about 1 micron to about 6 microns, about 1 micron to about 5 microns, about 1 micron to about 4 microns, about 2 microns to about 10 microns, about 3 microns to about 10 microns, about 4 microns to about 10 microns, about 4 microns to about 8 microns, about 5 microns to about 10 microns, or about 6 microns to about 10 microns, or any value between these ranges. In some embodiments the micronized solabegron particles may have an average particle size between a lower limit of about 0.1 microns, about 0.2 microns, about 0.3 microns, about 0.4 microns, about 0.5 microns, about 0.60 microns, about 0.70 microns, about 0.80 microns, about 0.90 microns, about 1 microns, about 2.0 microns, about 3 microns, about 4 microns, about 5 microns, about 6 microns, about 7 microns, about 8.0 microns, about 9 microns, about 10 microns, about 11 microns, about 12 microns, about 13 microns, about 14 microns, about 15 microns, about 16 microns, about 17 microns, about 18 microns, about 19 microns, about 20 microns, about 21 microns, about 22 microns, about 23 microns, about 24 microns, about 25 microns, about 26 microns, about 27 microns, about 28 microns, about 29 microns, and about 30 microns; and an upper limit of about 30 microns, about 29 microns, about 28 about microns, about 27 microns, about 26 microns, about 25 microns, about 24 microns, about 23 about microns, about 22 microns, about 21 microns, about 20 about microns, about 19 microns, about 18 about microns, about 17 microns, about 16 microns, about 15 microns, about 14 microns, about 13 microns, about 12 microns, about 11 microns, about 10 microns, about 9 microns, about 8 microns, about 7 microns, about 6 microns, about 5 microns, about 4 microns, about 3 microns, about 2 microns, about 1 micron, about 0.9 microns, about 0.8 microns, about 0.7 microns, about 0.6 microns, about 0.5 microns, about 0.4 microns, about 0.3 microns, about 0.2 microns, and about 0.1 microns. Specific examples include a particle size of about 0.1 micron to about 10 microns, a particle size of about 4 microns to about 8 microns, about 0.1 micron, about 1 micron, about 2 microns, about 3 microns, about 4 microns, about 5 microns, about 6 microns, about 7 microns, about 8 microns, about 9 microns, about 10 microns, about 11 microns, about 12 microns, about 13 microns, about 14 microns, about 15 microns, about 16 microns, about 17 microns, about 18 microns, about 19 microns, about 20 microns, about 21 microns, about 22 microns, about 23 microns, about 24 microns, about 25 microns, about 26 microns, about 27 microns, about 28 microns, about 29 microns, about 30 microns, and ranges in between these values.

In some embodiments, at least 99% of the solabegron particles, at least 98% of the solabegron particles, at least 97% of the solabegron particles, at least 96% of the solabegron particles, at least 95% of the solabegron particles, at least 94% of the solabegron particles, at least 93% of the solabegron particles, at least 90% of the solabegron particles, at least 85% of the solabegron particles, at least 80% of the solabegron particles, at least 75% of the solabegron particles, at least 70% of the solabegron particles, at least 60% of the solabegron particles, at least 50% or at least 10% of the solabegron particles in the pharmaceutical composition have a particle size from, about 0.1 micron to 30 microns, about 0.1 micron to about 25 microns, about 0.1 micron to about 20 microns, about 0.1 micron to about 10 microns, about 0.1 micron to about 8 microns, about 0.1 micron to about 6 microns, about 0.1 micron to about 4 microns, about 0.1 micron to about 2 microns, or about 0.1 micron to about 1 micron, about 1 micron to about 10 microns, about 1 micron to about 9 microns, about 1 micron to about 8 microns, about 1 micron to about 7 microns, about 1 micron to about 6 microns, about 1 micron to about 5 microns, about 1 micron to about 4 microns, about 2 microns to about 10 microns, about 3 microns to about 10 microns, about 4 microns to about 10 microns, about 5 microns to about 10 microns, or about 6 microns to about 10 microns, or any value between these ranges. Specific examples include about 0.1 micron to about 10 microns, about 4 microns to about 8 microns, about 0.1 micron, about 1 micron, about 2 microns, about 3 microns, about 4 microns, about 5 microns, about 6 microns, about 7 microns, about 8 microns, about 9 microns, about 10 microns, about 11 microns, about 12 microns, about 13 microns, about 14 microns, about 15 microns, about 16 microns, about 17 microns, about 18 microns, about 19 microns, about 20 microns, about 21 microns, about 22 microns, about 23 microns, about 24 microns, about 25 microns, about 26 microns, about 27 microns, about 28 microns, about 29 microns, about 30 microns, and ranges in between these values.

In some embodiments, at least 90% of the solabegron particles (D90) in the pharmaceutical composition have a particle size from about 0.1 micron to 30 microns, about 0.1 micron to about 25 microns, about 0.1 micron to about 20 microns, about 0.1 micron to about 10 microns, about 0.1 micron to about 8 microns, about 0.1 micron to about 6 microns, about 0.1 micron to about 4 microns, about 0.1 micron to about 2 microns, or about 0.1 micron to about 1 micron, about 1 micron to about 10 microns, about 1 micron to about 9 microns, about 1 micron to about 8 microns, about 1 micron to about 7 microns, about 1 micron to about 6 microns, about 1 micron to about 5 microns, about 1 micron to about 4 microns, about 2 microns to about 10 microns, about 3 microns to about 10 microns, about 4 microns to about 10 microns, about 5 microns to about 10 microns, or about 6 microns to about 10 microns, or any value between these ranges. In some embodiments, at least 90% of the solabegron particles (D90) in the pharmaceutical composition have a particle size between a lower limit of about 0.1 microns, about 0.2 microns, about 0.3 microns, about 0. microns, about 0.5 mg/kg microns, about 0.60 microns, about 0.70 microns, about 0.80 microns, about 0.90 microns, about 1 microns, about 2.0 microns, about 5 microns, about 8.0 microns, about 10 microns, about 13 microns, about 15 microns, about 18 microns, about 20 microns, about 23 mg/kg microns, about 25 microns, about 28 microns, and about 30 microns; and an upper limit of about 30 microns, about 28 about microns, about 23 about microns, about 20 about microns, about 18 about microns, about 15 microns, about 13 microns, about 10 microns, about 8 microns, about 5 microns, about 3 microns, about 1 micron, about 0.9 microns, about 0.8 microns, about 0.7 microns, about 0.6 microns, about 0.5 microns, about 0.4 microns, about 0.3 microns, about 0.2 microns, and about 0.1 microns. Specific examples include about 0.1 micron to about 10 microns, about 4 microns to about 8 microns, about 0.1 micron, about 1 micron, about 2 microns, about 3 microns, about 4 microns, about 5 microns, about 6 microns, about 7 microns, about 8 microns, about 9 microns, about 10 microns, about 11 microns, about 12 microns, about 13 microns, about 14 microns, about 15 microns, about 16 microns, about 17 microns, about 18 microns, about 19 microns, about 20 microns, 21 microns, about 22 microns, about 23 microns, about 24 microns, about 25 microns, about 26 microns, about 27 microns, about 28 microns, about 29 microns, about 30 microns, and ranges in between these values.

In some embodiments, at least 50% of the solabegron particles (D50) in the pharmaceutical composition have a particle size from about 0.1 micron to about 10 microns, about 0.1 micron to about 9 microns, about 0.1 micron to about 8 microns, about 0.1 micron to about 7 microns, about 0.1 micron to about 6 microns, about 0.1 micron to about 5 microns, about 0.1 micron to about 4 microns, about 0.1 micron to about 3 microns, about 0.1 micron to about 2 microns, or about 0.1 micron to about 1 micron, about 1 micron to about 5 microns, about 1 micron to about 4 microns, about 1 micron to about 3 microns, about 2 microns to about 5 microns, about 2 microns to about 4 microns, about 3 microns to about 4 microns, or any value between these ranges. In some embodiments, at least 50% of the solabegron particles (D50) in the pharmaceutical composition have a particle size between a lower limit of about 0.1 microns, about 0.2 microns, about 0.3 microns, about 0.4 microns, about 0.5 mg/kg microns, about 0.60 microns, about 0.70 microns, about 0.80 microns, about 0.90 microns, about 1 microns, about 2.0 microns, about 5 microns, about 8.0 microns, about 10 microns, about 13 microns, about 15 microns, about 18 microns, about 20 microns, about 23 mg/kg microns, about 25 microns, about 28 microns, and about 30 microns; and an upper limit of about 30 microns, about 28 about microns, about 23 about microns, about 20 about microns, about 18 about microns, about 15 microns, about 13 microns, about 10 microns, about 8 microns, about 5 microns, about 3 microns, about 1 micron, about 0.9 microns, about 0.8 microns, about 0.7 microns, about 0.6 microns, about 0.5 microns, about 0.4 microns, about 0.3 microns, about 0.2 microns, and about 0.1 microns. Specific examples include about 0.1 micron to about 10 microns, about 4 microns to about 8 microns, about 0.1 micron, about 1 micron, about 2 microns, about 3 microns, about 4 microns, about 5 microns, about 6 microns, about 7 microns, about 8 microns, about 9 microns, about 10 microns, about 11 microns, about 12 microns, about 13 microns, about 14 microns, about 15 microns, about 16 microns, about 17 microns, about 18 microns, about 19 microns, about 20 microns, 21 microns, about 22 microns, about 23 microns, about 24 microns, about 25 microns, about 26 microns, about 27 microns, about 28 microns, about 29 microns, about 30 microns, and ranges in between these values.

In some embodiments the micronized solabegron has a D95 of about 7 microns to about 10 microns; a D90 of about 6 microns to 8 microns, a D50 of about 3 microns to about 4 microns; a D10 of about 0.8 microns to about 1.3 microns and any combination thereof.

In some embodiments the micronized solabegron has a D95 of about 7 microns to about 10 microns; and a D90 of about 6 microns to 8 microns.

In some embodiments the micronized solabegron has a D95 of about 7 microns to about 10 microns; and a D50 of about 3 microns to about 4 microns.

In some embodiments the micronized solabegron has a D95 of about 7 microns to about 10 microns; and a D10 of about 0.8 microns to about 1.3 microns and any combination thereof.

In some embodiments the micronized solabegron has a D90 of about 6 microns to 8 microns, and a D50 of about 3 microns to about 4 microns.

In some embodiments the micronized solabegron has a D90 of about 6 microns to 8 microns, and a D10 of about 0.8 microns to about 1.3 microns

In some embodiments the micronized solabegron has a D50 of about 3 microns to about 4 microns; and a D10 of about 0.8 microns to about 1.3.

In some embodiments the micronized solabegron has the following particle size distribution:

| Percentile (D value) | Size μm |
|---|---|
| 10 (D10) | about 0.86 to about 1.27 |
| 20 (D20) | about 1.33 to about 1.95 |
| 30 (D30) | about 1.87 to about 2.67 |
| 40 (D40) | about 2.44 to about 3.22 |
| 50 (D50) | about 3.0 to about 3.91 |
| 60 (D60) | about 3.55 to about 4.53 |
| 70 (D70) | about 4.16 to about 5.22 |
| 80 (D80) | about 4.96 to about 6.13 |
| 90 (D90) | about 6.33 to about 7.65 |
| 95 (D95) | about 7.77 to about 9.27 |

or any combination thereof.

In some embodiments the micronized solabegron has the following particle size distribution:

| Percentile (D value) | Size μm |
|---|---|
| 10 (D10) | 0.957 |
| 20 (D20) | 1.515 |
| 30 (D30) | 2.139 |
| 40 (D40) | 2.757 |
| 50 (D50) | 3.36 |
| 60 (D60) | 4.00 |
| 70 (D70) | 4.74 |
| 80 (D80) | 5.69 |
| 90 (D90) | 7.29 |
| 95 (D95) | 8.95 |

In some embodiments the micronized solabegron has the following particle size distribution:

| Percentile (D value) | Size μm |
|---|---|
| 10 (D10) | 0.857 |
| 20 (D20) | 1.333 |
| 30 (D30) | 1.866 |
| 40 (D40) | 2.441 |
| 50 (D50) | 2.997 |
| 60 (D60) | 3.55 |
| 70 (D70) | 4.16 |
| 80 (D80) | 4.96 |
| 90 (D90) | 6.33 |
| 95 (D95) | 7.77 |

In some embodiments the micronized solabegron has the following particle size distribution:

| Percentile (D value) | Size μm |
|---|---|
| 10 (D10) | 1.266 |
| 20 (D20) | 1.954 |
| 30 (D30) | 2.66 |
| 40 (D40) | 3.32 |
| 50 (D50) | 3.91 |
| 60 (D60) | 4.53 |
| 70 (D70) | 5.22 |
| 80 (D80) | 6.13 |
| 90 (D90) | 7.65 |
| 95 (D95) | 9.27 |

In some embodiments, the effective amount of micronized solabegron or its pharmaceutically acceptable salt or derivative thereof present in the composition is about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, or about 140 mg

In some embodiments, the effective amount of micronized solabegron or its pharmaceutically acceptable salt or derivative thereof present in the composition is from about 50 mg to about 140 mg, about 50 mg to about 130 mg, about 50 mg to about 125 mg, about 50 mg to about 110 mg, about 50 mg to about 100 mg, about 50 mg to about 90 mg, about 50 mg to about 80 mg, about 50 mg to about 75 mg, or about 50 mg to about 60 mg, or any value between these ranges.

In some embodiments the effective amount of micronized solabegron or its pharmaceutically acceptable salt or derivative thereof present in the composition is about 75 mg to 125 mg. In some embodiments the effective amount of micronized solabegron or its pharmaceutically acceptable salt or derivative thereof present in the composition is about 100 mg to 125 mg. In some embodiments the effective amount of micronized solabegron or its pharmaceutically acceptable salt or derivative thereof, present in the composition is about 100 mg. In some embodiments the effective amount of micronized solabegron or its pharmaceutically acceptable salt or derivative thereof, present in the composition is about 125 mg.

In some embodiments, the effective amount of the micronized solabegron or its pharmaceutically acceptable salt or derivative thereof present in the composition is about 20 wt % to about 60 wt %, about 20 wt % to about 50 wt %, about 20 wt % to about 40 wt %, about 20 wt % to about 30 wt %, about 1 wt % to about 20 wt %, or about 1 wt % to about 10 wt % of the total weight of the composition, or any value between these ranges.

### Non-Micronized Solabegron Compositions

Non-micronized solabegron is solabegron with an average particle size greater than 30 microns, greater than 40 microns, greater than 50 microns, greater than 60 microns, greater than 70 microns, greater than 80 microns, greater than 90 microns greater than 100 microns and any value between these ranges. In some embodiments non-micronized solabegron has a D90 greater than 30 microns, greater than 40 microns, greater than 50 microns, greater than 60 microns, greater than 70 microns, greater than 80 microns, greater than 90 microns greater than 100 microns and any value between these ranges.

An example of non-micronized solabegron has the following particle size distribution:

| Percentile (D value) | Size μm |
|---|---|
| 10 (D10) | 10.7 |
| 50 (D50) | 38.6 |
| 90 (D90) | 76.1 |

In some embodiments, the composition comprises non-micronized solabegron. In some embodiments, the effective amount of non-micronized solabegron or its pharmaceutically acceptable salt or derivative thereof present in the composition is about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, or about 140 mg.

In some embodiments, the effective amount of non-micronized solabegron or its pharmaceutically acceptable salt or derivative thereof present in the composition is from about 50 mg to about 140 mg, about 50 mg to about 130 mg, about 50 mg to about 125 mg, about 50 mg to about 110 mg, about 50 mg to about 100 mg, about 50 mg to about 90 mg, about 50 mg to about 80 mg, about 50 mg to about 75 mg, or about 50 mg to about 60 mg, or any value between these ranges.

In some embodiments, the effective amount of non-micronized solabegron or its pharmaceutically acceptable salt or derivative thereof present in the composition is about 20 wt % to about 60 wt %, about 20 wt % to about 50 wt %, about 20 wt % to about 40 wt %, about 20 wt % to about 30 wt %, about 1 wt % to about 20 wt %, or about 1 wt % to about 10 wt % of the total weight of the composition, or any value between these ranges.

### Additional Therapeutic Agents

In some embodiments, the pharmaceutical composition further comprises one or more additional therapeutic agents selected from the group consisting of antimuscarinic agents, alpha adrenoceptor blockers, botulinum toxin, purinergics, cannabinoids, transient receptor potential (TRP) protein inhibitors, prostaglandins, percutaneous tibial nerve stimulators, sacral nerve stimulators, 5-alpha reductase inhibitors, phosphodiesterase-5 inhibitors, beta-3 adrenoreceptor agonists, and combination thereof.

In some embodiments, the antimuscarinic agent may be selected from tolterodine, oxybutynin, trospium, solifenacin, darifenacin, propiverine, fesoterodine, and pharmaceutically acceptable salts thereof. In some embodiments the antimuscarinic agent is tolterodine or trospium. In some embodiments the antimuscarinic agent is tolterodine. In some embodiments the antimuscarinic agent is trospium

In some embodiments, alpha adrenoceptor blockers may be selected from tamuslosin, alfuzosin, and silodosin and pharmaceutically acceptable salts thereof.

In some embodiments, 5-alpha reductase inhibitors may be selected from finasteride, dutasteride, epristeride, alfatradiol, and pharmaceutically acceptable salts thereof.

In some embodiments, phosphodiesterase-5 inhibitors may be selected from sildenafil, tadalafil, vardenafil, udenafil, avanafil and pharmaceutically acceptable salts thereof.

In some embodiments, beta-3 adrenoreceptor agonists may be selected from mirabegron, amibegron, ritobegron, vibegron, L-742,791, L-796,568, TRK-380, LY-368,842, Ro40-2148, and pharmaceutically acceptable salt thereof.

### Pharmaceutically Acceptable Excipients

In some embodiments, the pharmaceutical composition further comprises pharmaceutically acceptable excipients. Examples of pharmaceutically acceptable excipients that may be present in the composition include but not limited to fillers/vehicles, solvents/co-solvents, preservatives, antioxidants, suspending agents, surfactants, antifoaming agents, buffering agents, chelating agents, sweeteners, flavoring agents, binders, extenders, disintegrants, diluents, lubricants, fillers, wetting agents, glidants, and combinations thereof.

In some embodiments, exemplary fillers that may be present in the pharmaceutical composition include cellulose and cellulose derivatives such as microcrystalline cellulose; starches such as dry starch, hydrolyzed starch, and starch derivatives such as corn starch; cyclodextrin; sugars such as powdered sugar and sugar alcohols such as lactose, mannitol, sucrose and sorbitol; inorganic fillers such as aluminum hydroxide gel, precipitated calcium carbonate, carbonate, magnesium aluminometasilicate, dibasic calcium phosphate; and sodium chloride, silicon dioxide, titanium dioxide, titanium oxide, dicalcium phosphate dihydrate, calcium sulfate, alumina, kaolin, talc, or combinations thereof. Fillers may be present in the composition from about 20 wt % to about 65 wt %, about 20 wt % to about 50 wt %, about 20 wt % to about 40 wt %, about 45 wt % to about 65 wt %, about 50 wt % to about 65 wt %, or about 55 wt % to about 65 wt % of the total weight of the composition, or any value between these ranges.

In some embodiments, exemplary disintegrants that may be present in the pharmaceutical composition include starches, alginic acid, crosslinked polymers such as, e.g., crosslinked polyvinylpyrrolidone, croscarmellose sodium, potassium or sodium starch glycolate, clays, celluloses, starches, gums, or combinations thereof. Disintegrants may be present in the composition from about 1 wt % to about 10 wt %, about 1 wt % to about 9 wt %, about 1 wt % to about 8 wt %, about 1 wt % to about 7 wt %, about 1 wt % to about 6 wt %, or about 1 wt % to about 5 wt % of the total weight of the composition, or any value between these ranges.

In some embodiments, the pharmaceutical composition comprises binders, including but not limited to celluloses such as hydroxypropylcellulose, methyl cellulose, and hydroxypropylmethylcellulose; starches such as corn starch, pregelatinized starch, and hydroxpropyl starch; waxes and natural and synthetic gums such as acacia, tragacanth, sodium alginate; synthetic polymers such as polymethacrylates and polyvinylpyrrolidone; and povidone, dextrin, pullulane, agar, gelatin, tragacanth, macrogol, or combinations thereof. Binders may be present in the composition from about 0.5 wt % to about 5 wt %, about 0.5 wt % to about 4 wt %, about 0.5 wt % to about 3 wt %, about 0.5 wt % to about 2 wt %, or about 0.5 wt % to about 1 wt % of the total weight of the composition, or any value between these ranges.

In some embodiments, the pharmaceutical composition comprises wetting agents, including but not limited to oleic acid, glyceryl monostearate, sorbitan mono-oleate, sorbitan monolaurate, triethanolamine oleate, polyoxyethylene sorbitan mono-oleate, polyoxyethylene sorbitan monolaurate, sodium oleate, sodium lauryl sulfate, poloxamers, poloxamer 188, polyoxyethylene ethers, polyoxyethylene sorbitan fatty acid esters, polyoxyethylene fatty acid esters, polyethylene glycol fatty acid esters, polyoxyethylene hardened castor oil, polyoxyethylene alkyl ethers, polysorbates, cetyl alcohol, glycerol fatty acid esters (e.g., triacetin, glycerol monostearate, etc.), polyoxymethylene stearate, sodium lauryl sulfate, sorbitan fatty acid esters, sucrose fatty acid esters, benzalkonium chloride, polyethoxylated castor oil, and combinations thereof. Wetting agents may be present in the composition from about 0.1 wt % to about 1 wt %, about 0.1 wt % to about 2 wt %, about 0.1 wt % to about 3 wt %, about 0.1 wt % to about 4 wt %, or about 0.1 wt % to about 5 wt % of the total weight of the composition, or any value between these ranges.

In some embodiments, the pharmaceutical composition comprises lubricants, including but not limited to stearic acid, magnesium stearate, calcium hydroxide, talc, corn starch, sodium stearyl fumerate, alkali-metal and alkaline earth metal salts, waxes, boric acid, sodium benzoate, sodium acetate, sodium chloride, leucine, polyethylene glycol (PEG), a methoxypolyethylene glycol, propylene glycol, sodium oleate, glyceryl behenate, glyceryl palmitostearate, glyceryl benzoate, magnesium lauryl sulfate, sodium lauryl sulfate, and combinations thereof. Lubricants may be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition, or any value between these ranges.

In some embodiments, the pharmaceutical composition comprises glidants, including but not limited to colloidal silicon dioxide, talc, sodium lauryl sulfate, native starch, and combinations thereof. Glidants may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

In some embodiments, exemplary buffering agents that may be present in the composition include gluconate, lactate, citrate, acetate, phosphate, benzoate, carbonate salts, or combinations thereof. The buffering agent can be present in an amount sufficient to buffer the pH of the solution and minimize degradation of the active ingredients. Some buffering agents can also modulate active ingredient solubility in the liquid dosage form. The pH can be adjusted with a combination of two or more of these buffering agents, e.g., citric acid and sodium benzoate. Buffering agents may be present in the composition from about 1 wt % to about 10 wt %, about 1 wt % to about 9 wt %, about 1 wt % to about 8 wt %, about 1 wt % to about 7 wt %, about 1 wt % to about 6 wt %, or about 1 wt % to about 5 wt % of the total weight of the composition, or any value between these ranges.

In some embodiments, exemplary preservative agents that may be present in the composition include sodium benzoate, paraoxybenzoic acid esters, methyl, ethyl, butyl, and propyl parabens, chlorobutanol, benzyl alcohol, phenylethylalcohol, dehydroacetic acid, sorbic acid, benzalkonium chloride (BKC), benzethonium chloride, phenol, phenylmercuric nitrate, thimerosal, or combinations thereof. Preservative agents can be included in the liquid dosage form. The preservative agents can be in an amount sufficient to extend the shelf-life or storage stability, or both, of the liquid dosage form. Preservatives may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

In some embodiments, non-limiting examples of flavoring agents that may be present in the composition include synthetic flavor oils and flavoring aromatics and/or natural oils, extracts from plants leaves, flowers, fruits, and so forth and the like or any combinations thereof. These may include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil and the like or any combinations thereof. Also useful as flavors are vanilla, citrus oil, including lemon, orange, grape, lime and grapefruit, and fruit essences, including apple, banana, pear, peach, strawberry, raspberry, cherry, plum, pineapple, apricot, strawberry flavor, tutti-fruity flavor, mint flavor, or any combinations thereof. Flavoring agents may be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition, or any value between these ranges.

In some embodiments, exemplary antioxidants that may be present in the composition include flavonoids, anthocyanidins, anthocyanins, proanthocyanidins, or combinations thereof. Antioxidants may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

In some embodiments, exemplary sweetening agents that may be present in the composition include sorbitol, saccharin, acesulfame, e.g., acesulfame potassium, sucralose, xylitol, maltitol, sucrose, aspartame, fructose, neotame, glycerin, sodium saccharate, glycyrrhizin dipotassium, acesulfame K, mannitol, invert sugar, or combinations thereof. In some instances, a sweetening agent, such as one or more sucralose-containing components or saccharin-containing components, can be added to the pharmaceutical composition to modify the taste of the pharmaceutical composition. Sweetening agents may be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition, or any value between these ranges.

In some embodiments, the pharmaceutical compositions comprise surfactants selected from polysorbate 20, polysorbate 60, polysorbate 80, vitamin E TPGS, cremophor, solutol, poloxamer 121, poloxamer 124, poloxamer 181, poloxamer 188, poloxamer 237, poloxamer 331, poloxamer 338, poloxamer 407, poloxamer 407, Labrasol®, Labrafils®, Gelucire® 50/13, Gelucire® 44/14, Gelucire® 48/16, Gelucire® 55/18, Gelucire® 35/10, Gelucire® 48/09, lauroglycol, propylene glycol, polyethylene glycol, PEG 300, PEG 400, PEG 1000, Soluplus®, SDS, SLS, polyoxyl stearates, sorbitan esters, sucrose esters, stearic acid, cetyl alcohol, cetyl pyridinium chloride, docusate sodium, glyceryl monooleate, glyceryl monostearate, glyceryl palmitostearate, lecithin, oleic acid, and combinations thereof. In some embodiments, the surfactants may be present in the composition from about 0.1 wt % to about 30 wt %, about 0.1 wt % to about 25 wt %, about 0.1 wt % to about 20 wt %, about 0.1 wt % to about 15 wt %, about 0.1 wt % to about 10 wt %, or about 0.1 wt % to about 5 wt % of the total weight of the composition, or any value between these ranges. Specific examples include about 0.1 wt %, about 0.5 wt %, about 1 wt % about 5 wt %, about 10 wt %, about 20 wt %, about 25 wt %, or about 30 wt %.

In some embodiments, the pharmaceutical composition is a tablet and comprises a top coat, such as hydroxypropyl-methylcellulose coating or polyvinyl alcohol coating, and are available under the trade name Opadry™, such as Opadry White, Opadry II. Top coats may be present in the composition from about 1 wt % to about 10 wt %, about 1 wt % to about 9 wt %, about 1 wt % to about 8 wt %, about 1 wt % to about 7 wt %, about 1 wt % to about 6 wt %, or about 1 wt % to about 5 wt % of the total weight of the composition, or any value between these ranges.

In some embodiments, the pharmaceutical composition comprises micronized solabegron or a pharmaceutically acceptable salt or a derivative thereof, according to any embodiment described herein, and an excipient selected from the group consisting of, mannitol, poloxamer 188, methyl cellulose, croscarmellose sodium, magnesium stearate, colloidal silicon dioxide, and polyvinyl alcohol, or combinations thereof. In some embodiments, the pharmaceutical composition comprises micronized solabegron or a pharmaceutically acceptable salt or a derivative thereof, according to any embodiment described herein, mannitol, poloxamer 188, methyl cellulose, croscarmellose sodium, magnesium stearate, colloidal silicon dioxide, and polyvinyl alcohol, In some embodiments, the pharmaceutical composition comprises about 20 wt % to about 40 wt % of micronized solabegron HCl, about 45 wt % to about 65 wt % of mannitol, about 1 wt % to about 10 wt % croscarmellose sodium, about 0.5 wt % to about 5 wt % of methyl cellulose, about 0.1 wt % to about 1 wt % of poloxamer 188, about 0.1 wt % to about 5 wt % of magnesium stearate, about 0.05 wt % to about 1 wt % of colloidal silicon dioxide, and about 1 wt % to about 10 wt % of Opadry II.

In some embodiments, the pharmaceutical composition comprises: micronized solabegron or a pharmaceutically acceptable salt or a derivative thereof, according to any embodiment described herein and an excipient selected from the group consisting of sucrose, wheat starch, microcrystalline cellulose, talc, lactose monohydrate, calcium carbonate, titanium dioxide, stearic acid, croscarmellose sodium, povidone, polyethylene glycol 8000, colloidal silicon dioxide, ferric oxide, carboxymethylcellulose sodium, white wax, magnesium stearate, and carnauba wax or combinations thereof. In some embodiments, the pharmaceutical composition comprises: micronized solabegron or a pharmaceutically acceptable salt or a derivative thereof, according to any embodiment described herein, sucrose, wheat starch, microcrystalline cellulose, talc, lactose monohydrate, calcium carbonate, titanium dioxide, stearic acid, croscarmellose sodium, povidone, polyethylene glycol 8000, colloidal silicon dioxide, ferric oxide, carboxymethylcellulose sodium, white wax, magnesium stearate, and carnauba wax.

In some embodiments, the pharmaceutical composition comprises micronized solabegron or a pharmaceutically acceptable salt or a derivative thereof, according to any embodiment described herein and an excipient selected from the group consisting of colloidal anhydrous silica, calcium hydrogen phosphate dihydrate, cellulose microcrystalline, hypromellose, magnesium stearate, sodium starch glycolate (pH 3.0 to 5.0), stearic acid, and titanium dioxide or combinations thereof. In some embodiments, the pharmaceutical composition comprises micronized solabegron or a pharmaceutically acceptable salt or a derivative thereof, according to any embodiment described herein, colloidal anhydrous silica, calcium hydrogen phosphate dihydrate, cellulose microcrystalline, hypromellose, magnesium stearate, sodium starch glycolate (pH 3.0 to 5.0), stearic acid, and titanium dioxide

In some embodiments, the compositions disclosed herein may be formulated as tablets, capsules, granules, or powders. In some embodiments, the composition may be a tablet, a bi-layer tablet, a capsule, a multiparticulate, a drug coated sphere, a matrix tablet, or a multicore tablet.

For obtaining micronized solabegron particles, any known method in the art can be used. For example, solabegron particles of a composition according to the invention are dispersed in a liquid dispersion media in which the solabegron particles are poorly soluble and mechanical means is applied in the presence of grinding media to reduce the particle size of the composition to the desired effective average particle size. The particles can be reduced in size in the presence of one or more nonionic surface stabilizers. Alternatively, the particles can be contacted with one or more nonionic surface stabilizers after attrition. Other compounds, such as a diluent, can be added to the composition during the size reduction process. Dispersions can be manufactured continuously or in a batch mode.

The mechanical means applied to reduce the particle size of a composition according to the invention conveniently can take the form of a dispersion mill. Suitable dispersion mills include a ball mill, an attritor mill, a vibratory mill, jet mill, Fitz mill, and media mills such as a sand mill and a bead mill.

In some embodiments, tablets may be prepared using reagents and techniques readily available in the art and/or exemplary methods as described herein. Compressed tablets may be prepared by compressing in a suitable machine an active compound in a free-flowing form such as a powder or granules optionally mixed with a binder, lubricant, inert diluent, lubricating agent, surface-active agent or dispersing agent, together with the materials for forming the core. The core tablet may be capable of immediate release or delayed release.

### Immediate Release and Modified Release Compositions

In some embodiments, the micronized and non-micronized solabegron compositions disclosed herein are immediate release pharmaceutical compositions, modified release pharmaceutical compositions, or a combination thereof. In some embodiments, the immediate release pharmaceutical composition releases the solabegron within a short period of time after administration, typically less than about 4 hours, less than about 3.5 hours, less than about 3 hours, less than about 2.5 hours, less than about 2 hours, less than about 90 minutes, less than about 60 minutes, less than about 45 minutes, less than about 30 minutes, less than about 20 minutes, or less than about 10 minutes.

In some embodiments, the in-vitro dissolution rate of the immediate release pharmaceutical composition, when measured by the USP apparatus type II (paddle speed 50 rpm) at 37° C. is about 55%-65% of solabegron is released at about 10 minutes, about 65%-75% of solabegron is released at about 15 minutes, about 70%-80% of solabegron is released at about 20 minutes, about 75%-85% of solabegron is released at about 30 minutes, about 80%-90% of solabegron is released at about 45 minutes, or about 85%-95% of solabegron is released at about 60 minutes. In some embodiments, the dissolution media comprises about 900 ml solution containing 0.01 N HCl and 2% (w/v) poloxamer 188.

In some embodiments, immediate release pharmaceutical composition may be prepared using pharmaceutical processes namely by direct compression or by granulation processing and final tableting. The process may comprise the steps of initially forming a core comprising micronized solabegron followed by a top coat. The core may be formed by dispersing micronized solabegron with one or more excipients, such as mannitol, poloxamer 188, methyl cellulose, croscarmellose sodium, magnesium stearate, and colloidal silicon dioxide.

In some embodiments, an immediate release pharmaceutical composition of micronized solabegron tablet comprises the following:

TABLE 1
Immediate release pharmaceutical composition
of micronized solabegron tablet

| | mg/tablet | % w/w |
|---|---|---|
| Solabegron HCl (7.65 microns (D90)) | 136.1 | 30.24 |
| Mannitol | 254.8 | 56.62 |
| Croscarmellose sodium | 20.9 | 4.64 |
| Methyl cellulose | 12.3 | 2.73 |
| Poloxamer 188 | 3.0 | 0.66 |
| Magnesium stearate | 4.3 | 0.95 |
| Colloidal silicon dioxide | 1.3 | 0.288 |
| Opadry II(white) 85F18546 (low TiO2) | 17.3 | 3.84 |

In some embodiments, an immediate release composition of micronized solabegron tablet comprises the following:

TABLE 2
Immediate release pharmaceutical composition
of micronized solabegron tablet

| | mg/tablet | % w/w |
|---|---|---|
| Solabegron HCl (4.22-7.65 microns (D90)) | 125-140 | 27.7-31.1 |
| Mannitol | 240-270 | 53.3-60 |
| Croscarmellose sodium | 15-25 | 3.33-5.55 |
| Methyl cellulose | 10-15 | 2.22-3.33 |
| Poloxamer 188 | 2-5 | 0.44-1.11 |
| Magnesium stearate | 2-5 | 0.44-1.11 |
| Colloidal silicon dioxide | 0.5-3 | 0.11-0.66 |
| Opadry II (white) 85F18546 (low TiO2) | 15-20 | 3.33-4.44 |

In some embodiments, the immediate release composition may comprise micronized solabegron according to any embodiment described herein, from about 20 wt % to about 40 wt %, a filler from about 45 wt % to about 65 wt %, a disintegrant from about 1 wt % to about 10 wt %, a binder from about 0.5 wt % to about 5 wt %, a wetting agent from about 0.1 wt % to about 1 wt %, a lubricant from about 0.1 wt % to about 5 wt %, a glidant from about 0.05 wt % to about 1 wt %, and a film coat from about 1 wt % to about 10 wt %.

In some embodiments, the immediate release composition may comprise micronized solabegron according to any embodiment described herein, from about 27 wt % to about 31 wt %, a filler from about 53 wt % to about 60 wt %, a disintegrant from about 3 wt % to about 6 wt %, a binder from about 2 wt % to about 3.5 wt %, a wetting agent from about 0.4 wt % to about 1 wt %, a lubricant from about 0.4 wt % to about 1 wt %, a glidant from about 0.1 wt % to about 0.6 wt %, and a film coat from about 3 wt % to about 4.5 wt %.

In some embodiments, the immediate release composition comprises about 20 wt % to about 40 wt % of micronized solabegron according to any embodiment described herein, about 45 wt % to about 65 wt % of mannitol, about 1 wt % to about 10 wt % croscarmellose sodium, about 0.5 wt % to about 5 wt % of methyl cellulose, about 0.1 wt % to about 1 wt % of poloxamer 188, about 0.1 wt % to about 5 wt % of magnesium stearate, about 0.05 wt % to about 1 wt % of colloidal silicon dioxide, and about 1 wt % to about 10 wt % of Opadry II.

In some embodiments, the immediate release composition may comprise micronized solabegron according to any embodiment described herein, from about 25 wt % to about 35 wt %, mannitol from about 45 wt % to about 60 wt %, croscarmellose sodium from about 3 wt % to about 7 wt %, methylcellulose from about 2 wt % to about 4 wt %, poloxamer 188 from about 0.3 wt % to about 1 wt %, magnesium stearate from about 0.5 wt % to about 2 wt %, colloidal silicon dioxide from about 0.1 wt % to about 0.5 wt %, and Opadry II from about 2 wt % to about 5 wt %.

In some embodiments, the immediate release composition may comprise micronized solabegron according to any embodiment described herein, from about 29 wt % to about 31 wt %, mannitol from about 50 wt % to about 60 wt %, croscarmellose sodium from about 3 wt % to about 5 wt %, methylcellulose from about 2 wt % to about 3 wt %, poloxamer 188 from about 0.4 wt % to about 0.8 wt %, magnesium stearate from about 0.5 wt % to about 1.5 wt %, colloidal silicon dioxide from about 0.05 wt % to about 0.5 wt %, and Opadry II from about 2 wt % to about 4 wt %.

In some embodiments, the immediate release composition may comprise non-micronized solabegron according to any embodiment described herein, from about 20 wt % to about 40 wt %, a surfactant from about 0.1 wt % to about 25 wt %, a filler from about 45 wt % to about 65 wt %, a disintegrant from about 1 wt % to about 10 wt %, a binder from about 0.5 wt % to about 5 wt %, a wetting agent from about 0.1 wt % to about 1 wt %, a lubricant from about 0.1 wt % to about 5 wt %, a glidant from about 0.05 wt % to about 1 wt %, and a film coat from about 1 wt % to about 10 wt %.

In some embodiments, the modified release composition may release the micronized solabegron at a sustained or controlled rate over an extended period of time or may release the solabegron after a lag time after administration. For example, solabegron may be released from the composition 4 hours after administration, 8 hours after administration, 12 hours after administration, 16 hours after administration, or 24 hours after administration. Modified release compositions include, extended release, sustained release and delayed release compositions. In some embodiments, the modified release compositions may release about 10% of solabegron in about 2 hours, about 20% of solabegron in 2 hours, about 40% of solabegron in about 2 hours, about 50% of solabegron in about 2 hours, about 10% of solabegron in about 3 hours, about 20% of solabegron in 3 hours, about 40% of solabegron in about 3 hours, about 50% of solabegron in about 3 hours, about 10% of solabegron in about 4 hours, about 20% of solabegron in 4 hours, about 40% of solabegron in about 4 hours, about 50% of solabegron in about 4 hours, about 10% of solabegron in about 6 hours, about 20% of solabegron in 6 hours, about 40% of solabegron in about 6 hours, or about 50% of solabegron in about 6 hours.

In some embodiments, modified release compositions may comprise a matrix selected from microcrystalline cellulose, sodium carboxymethylcellulose, hydroxyalkylcelluloses such as hydroxy propyl methylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinyl acetate phthalate, polyalkylmethacrylates, polyvinyl acetate and mixtures thereof.

In some embodiments, the modified release composition may be a tablet. The tablet may comprise a core comprising micronized solabegron and a delayed release material compression coated on the core. In some embodiments, the delayed release material comprises heteropolysaccharide gum (e.g., xanthan gum), a homopolysaccharide gum (e.g., locust bean gum), and a saccharide (e.g., lactose, dextrose, mannitol, etc.). In certain embodiments, the gum(s) are wet granulated together with the optional saccharide(s) to form agglomerated particles comprising a mixture of, e.g., xanthan gum, locust bean gum and dextrose. In some embodiments, the delayed release material comprises hydroxypropylmethylcellulose, polymethacrylate-based copolymers, hydroxypropylcellulose, polyethylene oxide, alkylcelluloses such as methylcellulose and ethylcellulose, polyethylene glycol, polyvinylpyrrolidone, cellulose acetate, cellulose acetate butyrate, and the like.

In some embodiments, the compression coating delays the release of solabegron from the dosage form until after the dosage form has reached at least the mid small bowel after oral administration to humans. In certain embodiments, the initial release of solabegron from the dosage form does not occur until after entry of the dosage form into the distal small bowel. In certain alternate embodiments, the initial release of solabegron from the dosage form does not occur until after entry of the dosage form into the ileocaecal junction. In certain embodiments, the initial release of solabegron from the dosage form does not occur until after entry of the dosage form into the ascending colon. In certain embodiments, the initial release of solabegron from the dosage form does not occur until after entry of the dosage form into the hepatic flexure. In certain embodiments, the initial release of solabegron from the dosage form does not occur until after entry of the dosage form into the transverse colon.

In some embodiments, the micronized solabegron pharmaceutical compositions disclosed herein achieve a target area under the curve (herein after AUC) of about 5,000 ng·hr/mL to about 35,000 ng·hr/mL, about 10,000 ng·hr/mL to about 35,000 ng·hr/mL, about 15,000 ng·hr/mL to about 35,000 ng·hr/mL, about 20,000 ng·hr/mL to about 35,000 ng·hr/mL, about 15,000 ng·hr/mL to about 20,000 ng·hr/mL, or any value between these ranges over a 24-hour period. Specific examples include about 5,000 ng·hr/mL, about 10,000 ng·hr/mL, about 15,000 ng·hr/mL, about 17,000 ng·hr/mL, about 20,000 ng·hr/mL, about 25,000 ng·hr/mL, or about 35,000 ng·hr/mL over a 24-hour period. In some embodiments, the pharmaceutical compositions comprising micronized solabegron achieve a greater AUC when compared to a similar dosage composition prepared from non-micronized solabegron without surfactants, at least by 20%, at least by 30%, at least by 40%, at least by 50%, at least by 60%, at least by 70%, or at least by 80%.

Some embodiments describe a pharmaceutical composition comprising 75 mg to 125 mg of solabegron, wherein the solabegron is a combination of micronized solabegron having a particle size of about 0.1 microns to 30 microns and non-micronized solabegron having a particle size greater than 30 microns; wherein, when the pharmaceutical composition is administered to a subject, the pharmaceutical composition achieves an AUC₄₈ of about 22,239 ng·hr/mL or an AUC₄₈ of about 80%-125% of 22,239 ng·hr/mL.

In some embodiments the pharmaceutical composition comprises 75 mg to 125 mg of solabegron, wherein the solabegron is a combination of micronized solabegron having a particle size of about 0.1 microns to 30 microns and non-micronized solabegron having a particle size greater than 30 microns; wherein when the pharmaceutical composition is administered to a subject, the pharmaceutical composition achieves an AUC₂₄ of about 20,920 ng·hr/mL or an AUC₂₄ of about 80%-125% of an AUC of 20,920 ng·hr/mL.

In some embodiments the pharmaceutical composition comprises 75 mg to 125 mg of solabegron, according to one or more embodiments described herein; wherein when the pharmaceutical composition is administered to a subject, the pharmaceutical composition achieves an AUC₄₈ of about 22,239 ng·hr/mL or an AUC₄₈ of about 80%-125% of an AUC of 22,239 ng·hr/mL.

In some embodiments the pharmaceutical composition comprises 75 mg to 125 mg of solabegron, according to one or more embodiments described herein; wherein when the pharmaceutical composition is administered to a subject, the pharmaceutical composition achieves an AUC₂₄ of about 20,920 ng·hr/mL or an AUC₂₄ of about 80%-125% of 20,920 ng·hr/mL.

In some embodiments the pharmaceutical composition comprises 75 mg to 125 mg of micronized solabegron, according to any embodiment described herein; wherein when the pharmaceutical composition is administered to a subject, the pharmaceutical composition achieves an AUC of about 40% greater than the AUC achieved from the non-micronized solabegron of the same dose.

In some embodiments, the micronized solabegron pharmaceutical compositions disclosed herein achieve a target Cₘₐₓ of about 1 μg/mL to about 5.0 μg/mL, about 1 μg/mL to about 4.0 μg/mL, about 1 μg/mL to about 3.0 μg/mL, about 1 μg/mL to about 2.0 μg/mL, or about 1 μg/mL to about 1.5 μg/mL, or any value between these ranges. Specific examples include about 1.0 μg/mL, about 1.5 μg/mL, about 2.0 μg/mL, about 2.1 μg/mL, about 2.5 μg/mL, about 3.0 μg/mL, about 4.0 μg/mL, or about 5.0 μg/mL. In some embodiments, the pharmaceutical composition comprising micronized solabegron achieves a greater Cₘₐₓ when compared to a similar dosage composition prepared from non-micronized solabegron without surfactants, at least by 20%, at least by 30%, at least by 40%, at least by 50%, at least by 60%, at least by 70%, or at least by 80%.

In some embodiments, the pharmaceutical compositions comprising micronized and non-micronized solabegron and one or more surfactants may achieve pharmacokinetics similar to micronized solabegron pharmaceutical compositions disclosed herein. In some embodiments, the pharmaceutical compositions comprising micronized and non-micronized solabegron and one or more surfactants achieve a target area under the curve (herein after AUC) of about 5,000 ng·hr/mL to about 35,000 ng·hr/mL, about 10,000 ng·hr/mL to about 35,000 ng·hr/mL, about 15,000 ng·hr/mL to about 35,000 ng·hr/mL, about 20,000 ng·hr/mL to about 35,000 ng·hr/mL, about 15,000 ng·hr/mL to about 20,000 ng·hr/mL, or any value between these ranges over a 24 hour period. Specific examples include about 5,000 ng·hr/mL, about 10,000 ng·hr/mL, about 15,000 ng·hr/mL, about 17,000 ng·hr/mL, about 20,000 ng·hr/mL, about 25,000 ng·hr/mL, or about 35,000 ng·hr/mL over a 24-hour period.

In some embodiments, the pharmaceutical compositions comprising micronized and non-micronized solabegron and one or more surfactants achieves a target Cₘₐₓ of about 1 μg/mL to about 5.0 μg/mL, about 1 μg/mL to about 4.0 μg/mL, about 1 μg/mL to about 3.0 μg/mL, about 1 μg/mL to about 2.0 μg/mL, or about 1 μg/mL to about 1.5 μg/mL, or any value between these ranges. Specific examples include about 1.0 μg/mL, about 1.5 μg/mL, about 2.0 μg/mL, about 2.1 μg/mL, about 2.5 μg/mL, about 3.0 μg/mL, about 4.0 μg/mL, or about 5.0 μg/mL.

### Methods of Treatment

Embodiments of the present invention are also directed to methods of treating overactive bladder or one or more symptoms thereof in a subject in need thereof comprising administering to the subject a pharmaceutical composition comprising a therapeutically effective amount of micronized solabegron, according to any embodiment described herein. In some embodiments, micronized solabegron comprises at least 90% of the solabegron particles having a particle size of about 0.1 micron to 30 microns. In some embodiments, micronized solabegron comprises at least 90% of the solabegron particles having a particle size of about 0.1 micron to about 10 microns. In some embodiments, micronized solabegron comprises at least 90% of the solabegron particles having a particle size of about 4 microns to about 8 microns.

Embodiments of the present invention are also directed to methods of treating overactive bladder in a subject in need thereof comprising administering to the subject a pharmaceutical composition comprising a therapeutically effective amount of micronized solabegron, according to any embodiment described herein, wherein treating overactive bladder is measured by an increase in voided volume and one or more of the symptoms of overactive bladder is alleviated; wherein the one or more symptoms of overactive bladder is selected from the group consisting of urinary urgency, frequency of micturition, nocturia, and urgency urinary incontinence, or a combination thereof. In some embodiments, micronized solabegron comprises at least 90% of the solabegron particles having a particle size of about 0.1 micron to 30 microns. In some embodiments, micronized solabegron comprises at least 90% of the solabegron particles having a particle size of about 0.1 micron to about 10 microns. In some embodiments, micronized solabegron comprises at least 90% of the solabegron particles having a particle size of about 4 microns to about 8 microns. Embodiments of the present invention are also directed to methods of treating irritable bowel syndrome and similar gastrointestinal disorders in a subject in need thereof comprising administering to the subject a pharmaceutical composition comprising a therapeutically effective amount of micronized solabegron, according to any embodiment described herein. In some embodiments, micronized solabegron comprises at least 90% of the solabegron particles having a particle size of about 0.1 micron to 30 microns. In some embodiments, micronized solabegron comprises at least 90% of the solabegron particles having a particle size of about 0.1 micron to about 10 microns. In some embodiments, micronized solabegron comprises at least 90% of the solabegron particles having a particle size of about 4 microns to about 8 microns.

Embodiments of the present invention are also directed to methods of treating inflammatory bowel disease in a subject in need thereof comprising administering to the subject a pharmaceutical composition comprising a therapeutically effective amount of micronized solabegron, according to any embodiment described herein. In some embodiments, micronized solabegron comprises at least 90% of the solabegron particles having a particle size of about 0.1 micron to 30 microns. In some embodiments, micronized solabegron comprises at least 90% of the solabegron particles having a particle size of about 0.1 micron to about 10 microns. In some embodiments, micronized solabegron comprises at least 90% of the solabegron particles having a particle size of about 4 microns to about 8 microns.

Embodiments of the present invention are also directed to methods of treating overactive bladder or one or more symptoms thereof, in a subject in need thereof comprising orally administering to the subject a pharmaceutical composition comprising a therapeutically effective amount of about 75 mg to about 125 mg of micronized solabegron or a pharmaceutically acceptable salt or a derivative thereof, and a pharmaceutically acceptable excipient, wherein at least 90% of the solabegron particles have a particle size of about 0.1 micron to 30 microns. In some embodiments, at least 90% of the solabegron particles have a particle size of about 0.1 micron to about 10 microns. In some embodiments, the pharmaceutical composition is an immediate release composition. In some embodiments, the administration of the pharmaceutical composition is twice daily, orally.

In some embodiments, the symptoms of overactive bladder are selected from increased frequency of urinary urgency, nocturia, increase in urinary micturition frequency, urinary incontinence, and combination thereof.

In some embodiments, treating overactive bladder is measured by reduced frequency of urinary urgency, reduction in urinary micturition frequency, reduction in urinary incontinence episodes, reduction in urge urinary incontinence, percent dry rate (zero incontinence episodes), percent change from baseline in urge incontinence, increased voided volume, post-void residual volume, patient reported outcomes, and combination thereof.

Embodiments of the present invention are directed to methods of treating overactive bladder or one or more symptoms thereof, in a subject in need thereof, comprising orally administering to the subject a pharmaceutical composition comprising a therapeutically effective amount of about 125 mg of micronized solabegron or a pharmaceutically acceptable salt or a derivative thereof, and a pharmaceutically acceptable excipient, wherein at least 90% of the solabegron particles have a particle size of about 0.1 micron to 30 microns. In some embodiments, at least 90% of the solabegron particles have a particle size of about 0.1 micron to about 10 microns. In some embodiments, the pharmaceutical composition is an immediate release composition. In some embodiments, the administration of the pharmaceutical composition is twice daily, orally.

Embodiments of the present invention are also directed to methods of treating overactive bladder or one or more symptoms thereof, in a subject in need thereof, comprising orally administering to the subject a pharmaceutical composition comprising a therapeutically effective amount of about 100 mg of micronized solabegron or a pharmaceutically acceptable salt or a derivative thereof, and a pharmaceutically acceptable excipient, wherein at least 90% of the solabegron particles have a particle size of about 0.1 micron to about 10 microns. In some embodiments, the pharmaceutical composition is an immediate release composition. In some embodiments, the administration of the pharmaceutical composition is twice daily, orally.

Embodiments of the present invention are also directed to methods of treating overactive bladder or one or more symptoms thereof, in a subject in need thereof, comprising orally administering to the subject an immediate release pharmaceutical composition comprising a therapeutically effective amount of about 100 mg of micronized solabegron or a pharmaceutically acceptable salt or a derivative thereof, and a pharmaceutically acceptable excipient, wherein at least 90% of the solabegron particles have a particle size of about 0.1 micron to 30 microns. In some embodiments, 90% of the solabegron particles have a particle size of about 0.1 micron to about 10 microns. In some embodiments, the administration of the immediate release pharmaceutical composition is twice daily.

In some embodiments, the pharmaceutical composition comprising a therapeutically effective amount of micronized solabegron is more effective in treating overactive bladder or its symptoms when compared to a similar dosage pharmaceutical composition comprising a therapeutically effective amount of non-micronized solabegron without surfactants. In some embodiments, the pharmaceutical composition is an immediate release composition comprising about 100 mg of micronized solabegron and is administered orally twice daily.

In some embodiments, the pharmaceutical composition comprising a therapeutically effective amount of micronized solabegron is more effective in treating urinary incontinence when compared to a similar dosage pharmaceutical composition comprising a therapeutically effective amount of non-micronized solabegron without surfactants. In some embodiments, the pharmaceutical composition is an immediate release composition comprising about 100 mg of micronized solabegron and is administered orally twice daily.

In some embodiments, the pharmaceutical composition comprising a therapeutically effective amount of micronized solabegron is more effective in reducing micturition frequency when compared to a similar dosage pharmaceutical composition comprising a therapeutically effective amount of non-micronized solabegron without surfactants. In some embodiments, the pharmaceutical composition is an immediate release composition comprising about 100 mg of micronized solabegron and is administered orally twice daily.

In some embodiments, the pharmaceutical composition comprising a therapeutically effective amount of micronized solabegron is more effective in reducing urge urinary incontinence frequency when compared to a similar dosage pharmaceutical composition comprising a therapeutically effective amount of non-micronized solabegron without surfactants. In some embodiments, the pharmaceutical composition is an immediate release composition comprising about 100 mg of micronized solabegron and is administered orally twice daily.

In some embodiments, the pharmaceutical composition comprising a therapeutically effective amount of micronized solabegron is more effective in increasing percent dry rate when compared to a similar dosage pharmaceutical composition comprising a therapeutically effective amount of non-micronized solabegron without surfactants. In some embodiments, the pharmaceutical composition is an immediate release composition comprising about 100 mg of micronized solabegron and is administered orally twice daily.

In some embodiments, the pharmaceutical composition comprising a therapeutically effective amount of micronized solabegron is more effective in reducing the frequency of urinary urgency when compared to a similar dosage pharmaceutical composition comprising a therapeutically effective amount of non-micronized solabegron without surfactants. In some embodiments, the pharmaceutical composition is an immediate release composition comprising about 100 mg of micronized solabegron and is administered orally twice daily.

Embodiments of the present invention are also directed to methods of treating overactive bladder or one or more symptoms thereof, in a subject in need thereof, comprising orally administering to the subject a pharmaceutical composition comprising a therapeutically effective amount of micronized solabegron or a pharmaceutically acceptable salt or a derivative thereof, wherein the pharmaceutical composition achieves a target AUC₄₈ of about 17,000 ng·hr/mL to about 23,000 ng·hr/mL. In some embodiments, the therapeutically effective amount is about 100 mg of micronized solabegron and is administered twice daily. In some embodiments, the therapeutically effective amount is about 100 mg of micronized solabegron and is administered once daily.

Embodiments of the present invention are also directed to methods of treating overactive bladder or one or more symptoms thereof, in a subject in need thereof, comprising orally administering to the subject a pharmaceutical composition comprising a therapeutically effective amount of micronized solabegron or a pharmaceutically acceptable salt or a derivative thereof, wherein the pharmaceutical composition achieves a target AUC₄₈ of about 14,000 ng·hr/mL to about 29,000 ng·hr/mL. In some embodiments, the therapeutically effective amount is about 100 mg of micronized solabegron and is administered twice daily. In some embodiments, the therapeutically effective amount is about 100 mg of micronized solabegron and is administered once daily.

Embodiments of the present invention are also directed to methods of treating irritable bowel syndrome and similar gastrointestinal disorders in a subject in need thereof comprising orally administering to the subject a pharmaceutical composition comprising a therapeutically effective amount of about 50 mg to about 125 mg of micronized solabegron or a pharmaceutically acceptable salt or a derivative thereof, and a pharmaceutically acceptable excipient, wherein at least 90% of the solabegron particles have a particle size of about 0.1 micron to 30 microns. In some embodiments, 90% of the solabegron particles have a particle size of about 0.1 micron to about 10 microns. In some embodiments, the pharmaceutical composition is an immediate release composition. In some embodiments, the administration of the pharmaceutical composition is twice daily.

Embodiments of the present invention are also directed to methods of treating inflammatory bowel disease in a subject in need thereof comprising orally administering to the subject a pharmaceutical composition comprising a therapeutically effective amount of about 50 mg to about 125 mg of micronized solabegron or a pharmaceutically acceptable salt or a derivative thereof, and a pharmaceutically acceptable excipient, wherein at least 90% of the solabegron particles have a particle size of about 0.1 micron to 30 microns. In some embodiments, 90% of the solabegron particles have a particle size of about 0.1 micron to about 10 microns. In some embodiments, the pharmaceutical composition is an immediate release composition. In some embodiments, the administration of the pharmaceutical composition is twice daily. In some embodiments, inflammatory bowel disease comprises Crohn's disease and ulcerative colitis.

Embodiments of the present invention are also directed to methods of treating overactive bladder or one or more symptoms thereof, in a subject in need thereof, comprising administering to the subject a pharmaceutical composition comprising a therapeutically effective amount of non-micronized solabegron. In some embodiments, the composition comprises a therapeutically effective amount of about 50 mg to about 125 mg of non-micronized solabegron or a pharmaceutically acceptable salt or a derivative thereof, and a pharmaceutically acceptable excipient. In some embodiments, the non-micronized solabegron pharmaceutical composition further comprises one or more surfactants. In some embodiments, the pharmaceutical composition is an immediate release composition. In some embodiments, the administration of the pharmaceutical composition is twice daily.

Embodiments of the present invention are also directed to methods of treating irritable bowel syndrome and similar gastrointestinal disorders in a subject in need thereof comprising administering to the subject a pharmaceutical composition comprising a therapeutically effective amount of non-micronized solabegron. In some embodiments, the composition comprises a therapeutically effective amount of about 50 mg to about 125 mg of non-micronized solabegron or a pharmaceutically acceptable salt or a derivative thereof, and a pharmaceutically acceptable excipient. In some embodiments, the non-micronized solabegron pharmaceutical composition further comprises one or more surfactants. In some embodiments, the pharmaceutical composition is an immediate release composition. In some embodiments, the administration of the pharmaceutical composition is twice daily.

Embodiments of the present invention are also directed to methods of treating inflammatory bowel disease in a subject in need thereof comprising administering to the subject a pharmaceutical composition comprising a therapeutically effective amount of non-micronized solabegron. In some embodiments, the composition comprises a therapeutically effective amount of about 50 mg to about 125 mg of non-micronized solabegron or a pharmaceutically acceptable salt or a derivative thereof, and a pharmaceutically acceptable excipient. In some embodiments, the non-micronized solabegron pharmaceutical composition further comprises one or more surfactants. In some embodiments, the pharmaceutical composition is an immediate release composition. In some embodiments, the administration of the pharmaceutical composition is twice daily.

In some embodiments, the compositions disclosed herein may be administered once, as needed, once daily, twice daily, three times a day, once a week, twice a week, every other week, every other day, or the like for one or more dosing cycles. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks. It will be understood that the specific dose level and frequency of dosage for any particular subject can be varied and will depend upon a variety of factors including the species, age, body weight, general health, gender and diet of the subject, the mode and time of administration, rate of excretion, drug combination, and severity of the particular condition.

In some embodiments, when the composition is administered twice daily, the time period between administration of the first dose and the second dose is about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, or about 18 hours.

In some embodiments, the b.i.d. administration of an immediate release solid oral dosage form comprising about 100 mg micronized solabegron has a time interval between doses of 5-15 hours. In another embodiment, the b.i.d. administration of an immediate release solid oral dosage form comprising about 100 mg micronized solabegron has a time interval between doses of 6-16 hours. In another embodiment, the b.i.d. administration of an immediate release solid oral dosage form comprising about 100 mg micronized solabegron has a time interval between doses of 5-10 hours. In another embodiment, the b.i.d. administration of an immediate release solid oral dosage form comprising about 100 mg micronized solabegron has a time interval between doses of 6-12 hours.

In some embodiments, the pharmaceutical compositions disclosed herein are administered once daily. In some embodiments, the pharmaceutical composition comprises 100 mg of micronized solabegron and is administered once daily. In some embodiments, the pharmaceutical composition is an immediate release composition, a modified release composition, or a combination thereof.

Embodiments of the present invention are also directed to methods of treating overactive bladder or one or more symptoms thereof, in a subject in need thereof, comprising orally administering to the subject a pharmaceutical composition comprising a therapeutically effective amount of micronized solabegron or a pharmaceutically acceptable salt or a derivative thereof, wherein the pharmaceutical composition achieves a target AUC of about 5,000 ng·hr/mL to about 35,000 ng·hr/mL, and a Cₘₐₓ of about 1 μg/mL to about 5.0 μg/mL. In some embodiments, the pharmaceutical composition achieves a target AUC of about 17,000 ng·hr/mL. In other embodiments, the pharmaceutical composition achieves a Cₘₐₓ of 2.1 μg/mL. In some embodiments, the pharmaceutical composition comprises about 100 mg of micronized solabegron and is administered twice daily. In some embodiments, the pharmaceutical composition comprises about 100 mg of micronized solabegron and is administered once daily.

Embodiments of the present invention are also directed to methods of treating overactive bladder or one or more symptoms thereof, in a subject in need thereof, comprising orally administering to the subject a pharmaceutical composition comprising a therapeutically effective amount of non-micronized solabegron or a pharmaceutically acceptable salt or a derivative thereof, and one or more surfactants, wherein the pharmaceutical composition achieves a target AUC of about 5,000 ng·hr/mL to about 35,000 ng·hr/mL, and a Cₘₐₓ of about 1 μg/mL to about 5.0 μg/mL. In some embodiments, the pharmaceutical composition achieves a target AUC of about 17,000 ng·hr/mL. In other embodiments, the pharmaceutical composition achieves a Cₘₐₓ of 2.1 μg/mL. In some embodiments, the pharmaceutical composition comprises about 100 mg of non-micronized solabegron and is administered twice daily. In some embodiments, the pharmaceutical composition comprises about 100 mg of non-micronized solabegron and is administered once daily.

The pharmaceutical compositions of the present application can be administered for any of the uses described herein by any suitable means, for example, orally, sublingually, or bucally. The present compositions can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compositions can also be administered liposomally.

In some embodiments, methods of treating overactive bladder or symptoms thereof may further comprise administering a therapeutically effective amount of one or more additional therapeutic agents. In some embodiments, the one or more additional therapeutic agents may be administered prior to, simultaneously with, or following the administration of the pharmaceutical composition comprising solabegron. In some embodiments, the one or more additional therapeutic agents may be present along with solabegron in the same composition. In some embodiments, the one or more additional therapeutic agents may be antimuscarinic agents, alpha adrenoceptor blockers, botulinum toxin, purinergics, cannabinoids, transient receptor potential (TRP) protein inhibitors, prostaglandins, percutaneous tibial nerve stimulation, 5-alpha reductase inhibitors, phosphodiesterase-5 inhibitors, beta-3 adrenoreceptor agonists, and combination thereof. In some embodiments the additional therapeutic agents is trospium or tolterodine.

### EXAMPLES

### Example 1: Micronized Solabegron Tablet Formulations

Table 3 shows 125 mg and 175 mg tablet compositions of micronized solabegron.

TABLE 3
Composition of 125 mg and 175 mg immediate
release micronized solabegron tablets

| | Potency | | | | |
|---|---|---|---|---|---|
| | 125 mg | | 175 mg | | |
| | mg/ | % | mg/ | % | |
| Component | tablet | w/w | tablet | w/w | Function |
| Core Tablet: | | | | | |
| Solabegron HCl¹ | 136.1 | 30.2 | 190.5 | 42.3 | Active |
| Mannitol² | 254.8 | 56.6 | 189.4 | 42.0 | Filler |
| Croscarmellose Sodium | 20.9 | 4.6 | 25.8 | 5.7 | Disintegrant |
| Methyl Cellulose | 12.3 | 2.7 | 17.2 | 3.8 | Binder |
| Poloxamer 188 (Lutrol F 68) | 3.0 | 0.7 | 4.2 | 0.9 | Wetting |
| | | | | | Agent |
| Magnesium Stearate | 4.3 | 1.0 | 4.3 | 1.0 | Lubricant |
| Colloidal Silicon Dioxide | 1.3 | 0.3 | 1.3 | 0.3 | Glidant |
| Purified Water³ | — | — | — | — | Vehicle |
| Core Tablet Weight | 433.3 | | 433.3 | | |
| Film Coating: | | | | | |
| Opadry II (white) 85F18546 | 17.3 | 4.0 | 17.3 | 4.0 | Film Coat |
| | | | | | Polymer |
| Purified Water³ | — | — | — | — | Vehicle |
| Total Coated Tablet Weight | 450.1 | | 450.1 | | |

¹Equivalent to 125 and 175 mg solabegron free base
²Amount of mannitol may be adjusted to maintain the tablet core weight
³Removed during processing

Comparison of formulations of non-micronized and micronized solabegron tablet is shown in Table 4. The excipients used in the micronized formulation were identical to non-micronized solabegron formulations with the exception of the two minor differences list below. The differences in the excipients utilized for the micronized formulation had no impact on in-vitro tablet performance.
1. The Poloxamer P188, Lutrol F 68, included in the micronized formulation is the standard particle sized material as opposed to the “micro” material used historically. As Poloxamer is only 1% of the formulation, this change did not alter processability or dissolution performance.
2. Low TiO² Opadry II (white), 85F18546, was included in micronized formulation replacing Opadry White® OY-S-28876. Low TiO² Opadry II was chosen based upon better resistance to scuffing during processing.

TABLE 4
Comparison of excipients used in non-micronized and micronized solabegron tablets

| Excipients used in non- | Excipients used in | | |
|---|---|---|---|
| micronized solabegron | micronized solabegron | | |
| 100 mg tablet | 125/175 mg tablet | | |
| Description | Description | Comparison | Function |
| Mannitol, Pearlitol 160C, | Mannitol, Pearlitol 160C, | Same | Filler |
| EP & USP & JP, NOA | EP & USP & JP, NOA | | |
| Methylcellulose, | Methylcellulose, | Same | Binder |
| Methocel A15 Premium | Methocel A15 Premium | | |
| LV, EP & USP & JP, | LV, EP & USP & JP, | | |
| NOA | NOA | | |
| Poloxamer 188, Lutrol F | Poloxamer 188, Lutrol F | Same material grade | Wetting Agent |
| 68, Micro EP & USP, | 68, EP & USP, NOA | with larger particle | |
| NOA | | size. Did not slow | |
| | | dissolution rate | |
| Croscarmellose sodium, | Croscarmellose sodium, | Same | Disintegrant |
| Ac-Di-Sol, EP & USP & | Ac-Di-Sol, EP & USP & | | |
| JP, NOA | JP, NOA | | |
| Magnesium Stearate, | Magnesium Stearate, | Same | Lubricant |
| N/A EP & USP, NOA | N/A EP & USP, NOA | | |
| Colloidal Silicon | Colloidal Silicon Dioxide, | Same | Glidant |
| Dioxide, Cab-o-sil M- 5- | Cab-o-sil M- 5-P, EP & | | |
| P, EP & USP, NOA | USP, NOA | | |
| Opadry Film Coating, | Opadry II (white) | Low titanium | Film Coat |
| White/OY-S- 28876, | 85F18546, Non | dioxide coating | |
| Non Pharmacopoeial, | Pharmacopoeial, NOA | used to decrease | |
| NOA | | surface scuffing | |

### Example 2: Dissolution Profiles of Micronized and Non-Micronized Solabegron

The dissolution of various solabegron tablets were determined in accordance with the current USP<711> on dissolution using the USP Apparatus 2, and the parameters are shown in Table 5.

TABLE 5
Dissolution parameters

| | |
|---|---|
| Apparatus | USP Apparatus 2 (paddles) |
| Paddle Speed | 50 rpm |
| Dissolution Medium | 0.01N Hydrochloric Acid containing |
| | 2% (w/v) Poloxamer 188 |
| Dissolution Medium | 900 mL |
| Temperature | 37.0° = 0.5° C. |
| Detection | HPLC at 242 nm |

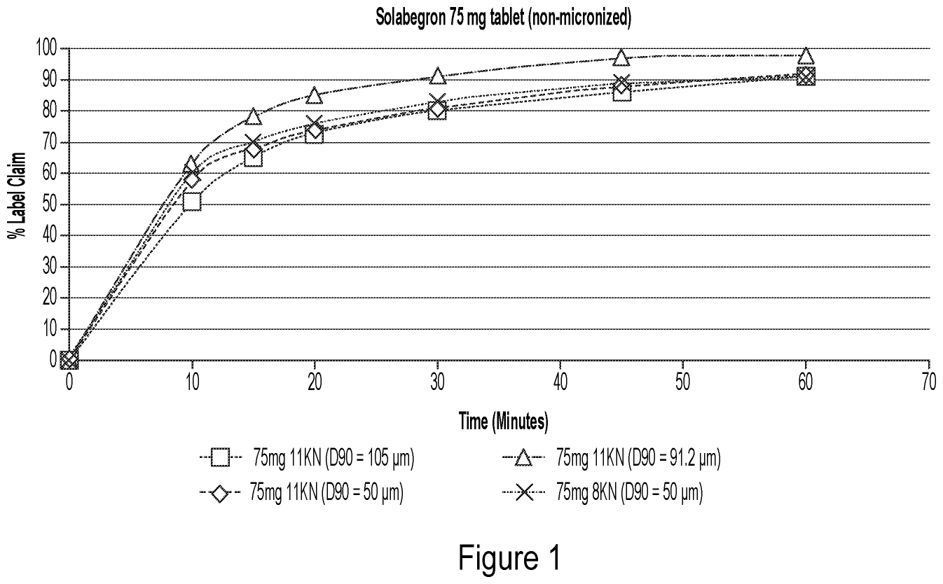

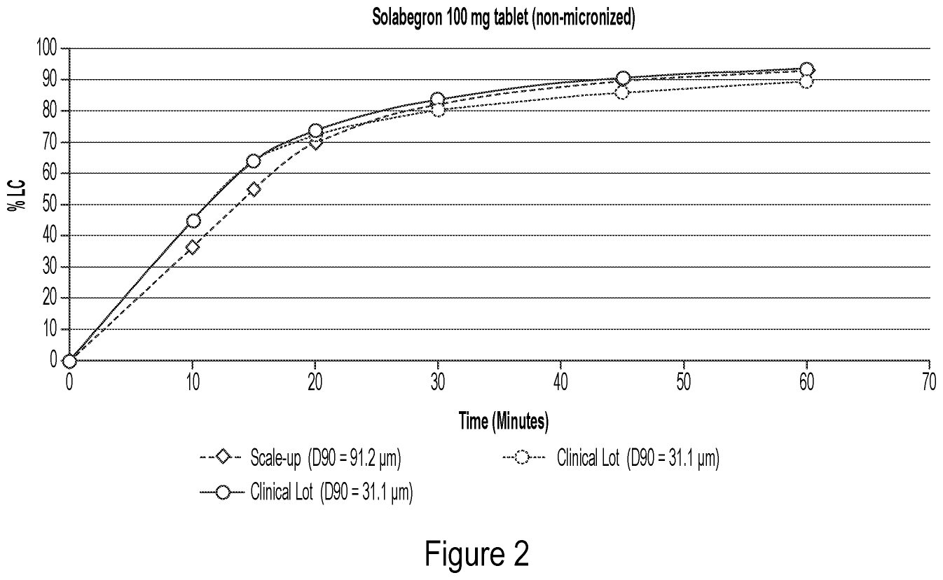

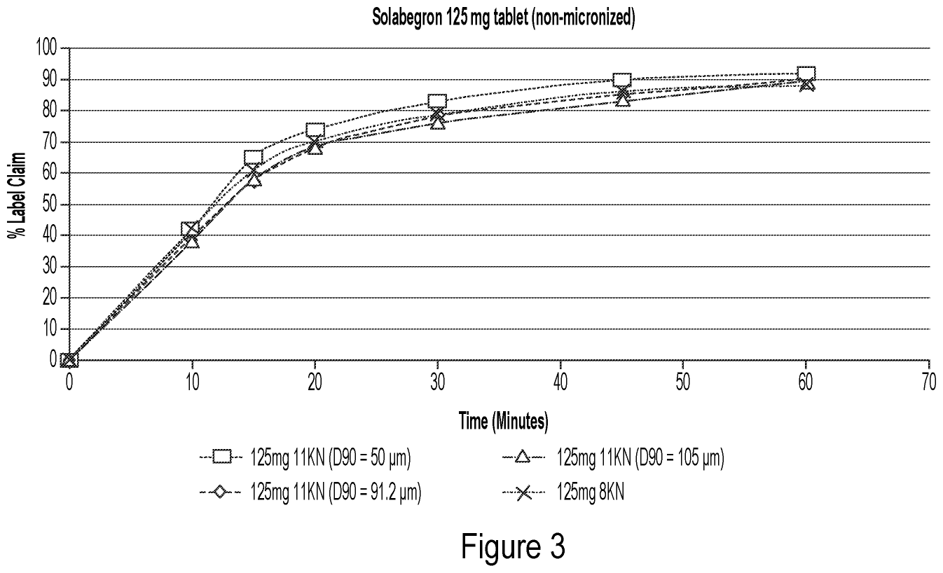

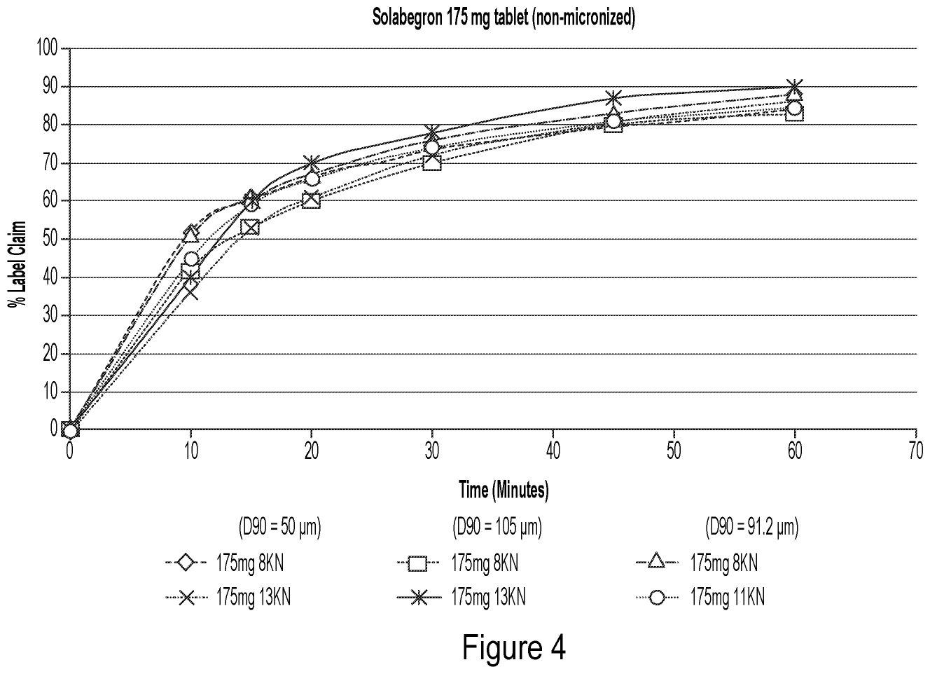

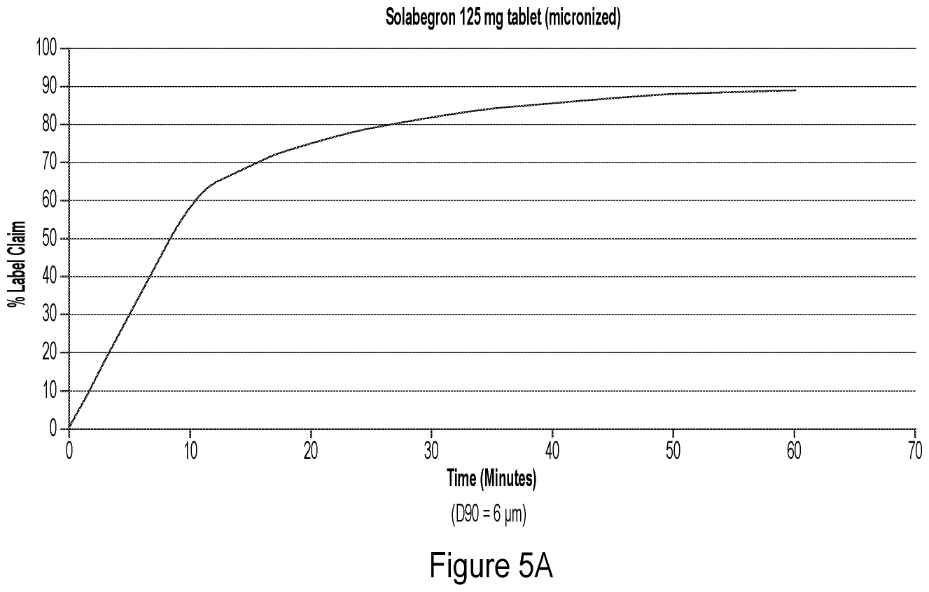

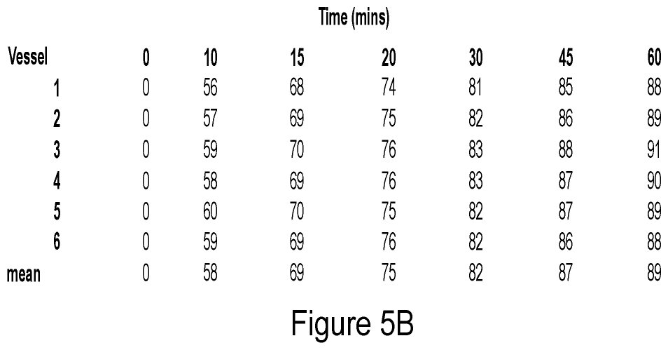

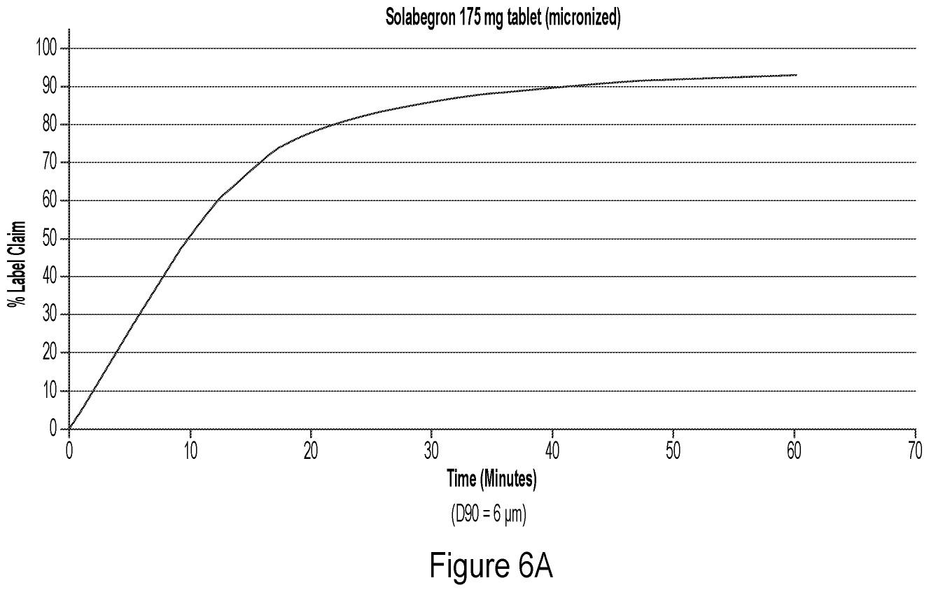

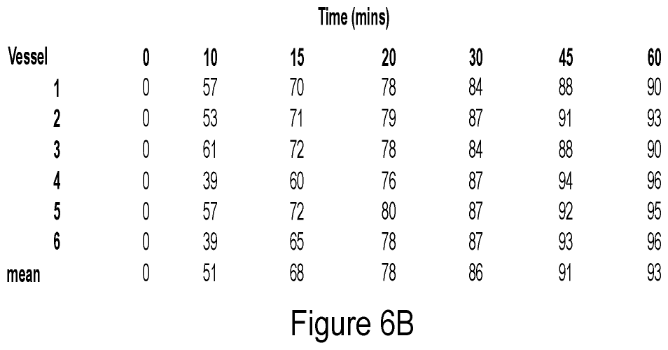

The dissolution profile of all the tablet formulations is shown in FIGS. 1-6.

### Example 3: Comparison of Dissolution Profiles of Micronized and Non-Micronized 125 mg Solabegron Tablet

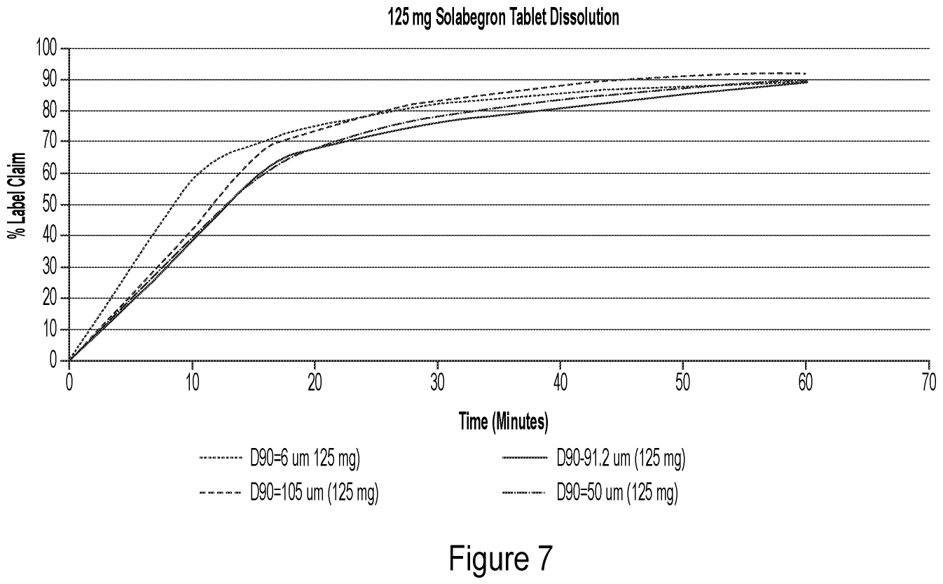

The dissolution of 125 mg micronized and 125 mg non-micronized solabegron tablets were determined using USP Type 2 apparatus and the parameters as described in Table 5. Table 6 shows the dissolution rate at various time points and the same is also illustrated in a graph in FIG. 7. The dissolution profiles of micronized and non-micronized tablets were similar, except for small difference in the dissolution rate at 10 min time-period.

TABLE 6
Comparison of dissolution profiles of micronized
and non-micronized 125 mg solabegron tablets

| | | Time (min) | | | | | |
|---|---|---|---|---|---|---|---|
| Tablet | 0 | 10 | 15 | 20 | 30 | 45 | 60 |
| Non-micronized Solabegron | 0 | 38 | 58 | 68 | 76 | 83 | 89 |
| (D90 = 91.2 μm) | | | | | | | |
| Non-micronized Solabegron | 0 | 42 | 65 | 74 | 83 | 90 | 92 |
| (D90 = 105 μm) | | | | | | | |
| Non-micronized Solabegron | 0 | 39 | 57.2 | 68 | 78.3 | 85.2 | 89.7 |
| (D90 = 50 μm) | | | | | | | |
| Micronized Solabegron | 0 | 58 | 69 | 75 | 82 | 87 | 89 |
| (D90 = 6 μm) | | | | | | | |

### Example 4: Comparison of Dissolution Profiles of Micronized and Non-Micronized 175 mg Solabegron Tablet

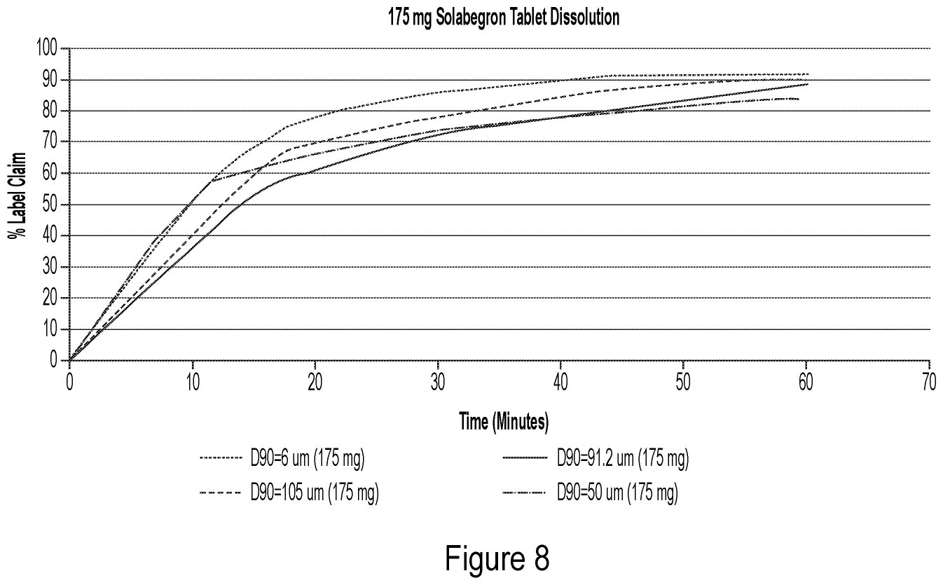

The dissolution of 175 mg micronized and 175 mg non-micronized solabegron tablets were determined using USP Type 2 apparatus and the parameters as described in Table 5. Table 7 shows the dissolution rate at various time points and the same is also illustrated in a graph in FIG. 8. The dissolution profiles of micronized and non-micronized tablets were similar at all intervals.

TABLE 7
Comparison of dissolution profiles of micronized
and non-micronized 175 mg solabegron tablets

| | | Time (min) | | | | | |
|---|---|---|---|---|---|---|---|
| Tablet | 0 | 10 | 15 | 20 | 30 | 45 | 60 |
| Non-micronized | 0 | 36 | 53 | 61 | 72 | 81 | 88 |
| Solabegron | | | | | | | |
| (D90 = 91.2 μm) | | | | | | | |
| Non-micronized | 0 | 40 | 60 | 70 | 78 | 87 | 90 |
| Solabegron | | | | | | | |
| (D90 = 105 μm) | | | | | | | |
| Non-micronized | 0 | 51.8 | 60.7 | 66.3 | 73.5 | 79.7 | 83.5 |
| Solabegron | | | | | | | |
| (D90 = 50 μm) | | | | | | | |
| Micronized | 0 | 51 | 68 | 78 | 86 | 91 | 93 |
| Solabegron | | | | | | | |
| (D90 = 6 μm) | | | | | | | |

### Example 5: Evaluation of the Efficacy of Micronized Solabegron Tablets

A Phase 2b, multicenter, randomized, double-blind, placebo-controlled, parallel-group study designed to evaluate the efficacy, safety, and tolerability of micronized solabegron tablet (solabegron D90 of about 4.22-7.65 microns) administered twice daily for 12 weeks to adult female subjects. Eligible female subject with overactive bladder symptoms (frequency, urgency, and predominantly urgency incontinence) for at least 6 months were selected for the study. Eligible subjects who met the pre-specified criteria were randomized 1:1:1 to receive micronized solabegron immediate release tablets, low dose or high dose, or matching placebo. The objectives of the study were: (a) to evaluate the efficacy of micronized solabegron immediate release tablets, 125 mg or 175 mg administered twice daily to adult female subjects with OAB; ((b) to evaluate the safety and tolerability of micronized solabegron immediate release tablets 125 mg or 175 mg administered twice daily to adult female subjects with OAB.

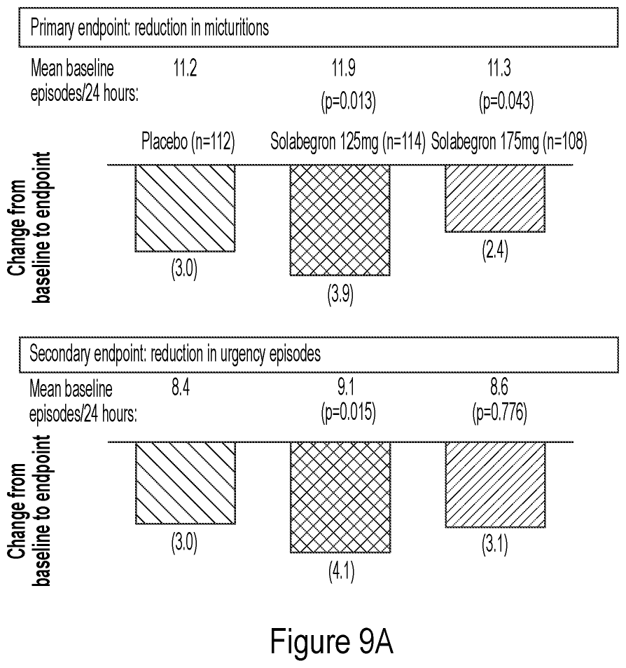

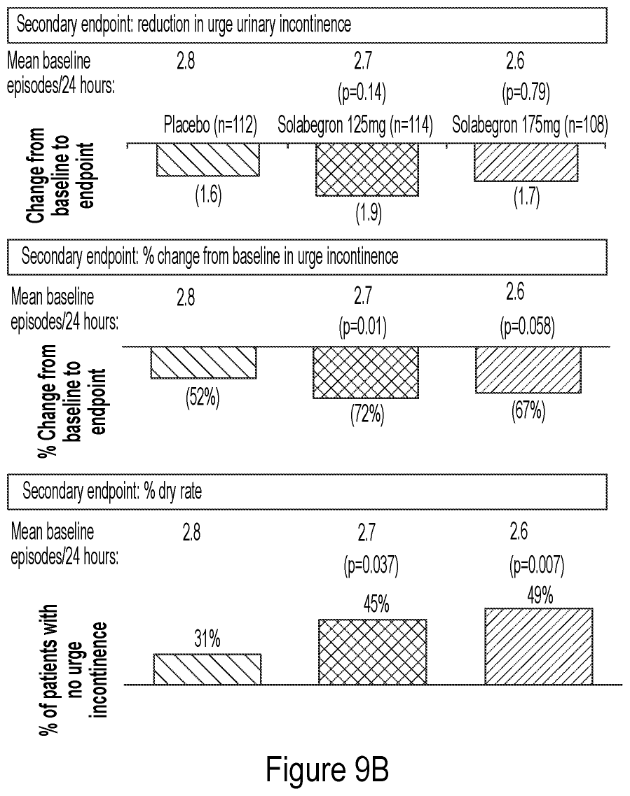

The primary efficacy endpoint for this study was change from baseline in mean number of micturitions per 24 h at week 12 in the MITT (Primary Analysis) population. Twice daily treatment with micronized solabegron 125 mg produced a mean reduction from baseline at week 12 of nearly 4 micturitions (−3.86) per 24 h. The comparison between the micronized solabegron 125 mg and placebo groups was statistically significant (placebo-corrected reduction=−0.82; p=0.0134). In contrast, the mean reduction in micturitions in the placebo group exceeded the mean reduction from baseline for the solabegron 175 mg group at week 12 (placebo-corrected increase; p=0.0428). Results are summarized for the MITT (Primary Analysis) Population using LOCF in Table 8 and in FIGS. 9A and 9B.

TABLE 8
Change from Baseline in Mean Number of Micturitions
per 24 Hours at Week 12, MITT Population Using LOCF

| | | | | | Mean | |
|---|---|---|---|---|---|---|
| | | | | | Difference | |
| | | | | | from | |
| Visit | Treatment | n | Mean | SD | Placebo | p-value |
| Baseline | Placebo | 112 | 11.22 | 2.528 | — | — |
| | Micronized | 114 | 11.87 | 2.807 | — | — |
| | Solabegron | | | | | |
| | 125 mg tablet | | | | | |
| | Micronized | 108 | 11.26 | 2.351 | — | — |
| | Solabegron | | | | | |
| | 175 mg tablet | | | | | |
| Week 12 | Placebo | 112 | −3.04 | 2.334 | — | — |
| | Micronized | 114 | −3.86 | 2.892 | −0.82 | 0.0134 |
| | Solabegron | | | | | |
| | 125 mg tablet | | | | | |
| | Micronized | 108 | −2.36 | 2.127 | 0.68 | 0.0428 |
| | Solabegron | | | | | |
| | 175 mg tablet | | | | | |

Abbreviations:
ANOVA = Analysis of Variance; LOCF = Last Observation Carried Forward; MITT = Modified Intent to Treat; n = number of observations; SD = standard deviation.
P-values were obtained from ANOVA model with contrast between each active treatment group and placebo.
ANOVA Model was: Change from Baseline value = Treatment.
If the visit data were missing, the last available post-baseline data were used.
Subjects with only 1 qualifying post-Baseline diary day were excluded.

Further, the micronized solabegron 125 mg and 175 mg groups were associated with 72% and 67% reductions, respectively, in urgency incontinence episodes from Baseline to Week 12. Statistically significant differences compared with placebo were apparent for the solabegron 125 mg and 175 mg groups at Week 8.

Statistically significant differences in the mean change from baseline for urgency assessments for the micronized solabegron 125 mg and 175 mg groups were observed compared with the placebo group at Week 12. Statistically significant differences were also demonstrated for the solabegron 175 mg and placebo groups at Weeks 4 and 8.

Statistically significant reductions from baseline in the mean number of urgency episodes per 24 h were demonstrated in the micronized solabegron 125 mg group in comparison to the placebo group at Weeks 8 and 12. The reduction in urgency episodes from Baseline was somewhat higher in the PP analysis, which demonstrated significant reductions compared with the placebo group for the micronized solabegron 125 mg and micronized solabegron 175 mg groups at Week 8 and for the micronized solabegron 125 mg group at Week 12.

Statistically significant reductions from baseline in % dry rate were also demonstrated by micronized solabegron 125 mg and 175 mg groups.

### Example 6: Comparison of Pharmacokinetics and Clinical Efficacy of Micronized and Non-Micronized Solabegron Tablets

A comparison of Phase 2 proof-of-concept study conducted with non-micronized solabegron. (Ohlstein, et. al., “A multicenter, double-blind, randomized, placebo-controlled trial of the β-adrenoceptor agonist solabegron for overactive bladder”, European Urology, 62(5): 834-840 Jun. 5, 2012) and the Phase 2 trial (Example 9) supports the significant and meaningful impact the micronization of the solabegron in the pharmaceutical composition unexpectedly had on the pharmacokinetics in patients with OAB.

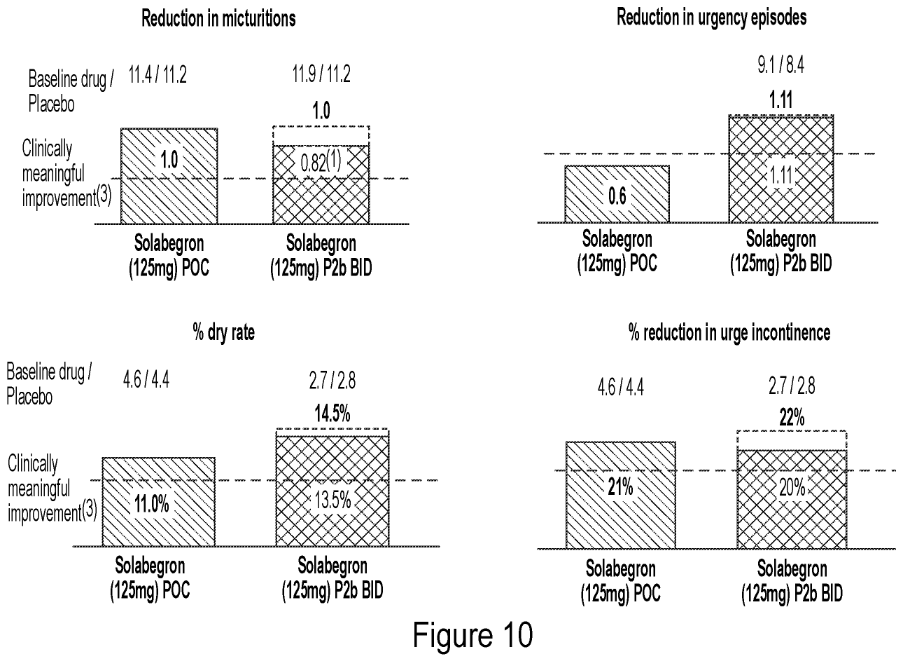

As shown in Table 9 and FIG. 10, 125 mg micronized solabegron tablet (D90 4.22-7.65 microns) in the Example 5 Study exhibited increased mean AUC (24 hrs.) and mean Cmax when compared to the non-micronized tablet.

TABLE 9
Pharmacokinetics and clinical efficacy comparison after dosing
125 mg non-micronized and micronized solabegron tablets

| | Mean Cₘₐₓ | Mean AUC (24 hrs) |
|---|---|---|
| Dose | (ng/mL) | (ng · hr/mL) |
| 125 mg (34.6 micronS D90 | 1680 | 14,988 |
| particle size) non-micronized | | |
| 125 mg (4.22-7.65 microns D90 | 2764 | 20,920 |
| particle size) | | |

Surprisingly, treatment with the micronized solabegron tablets resulted in an about a 40% increase in exposure (AUC).

The greater exposure levels achieved by the micronized formulation, allows for a reduction in the amount (dose) of solabegron needed to be administered to the patient. For example, based on the data presented in Table 9, for the 125 mg micronized solabegron formulation, a 100 mg dose of solabegron is predicted to provide better exposure than that of the 125 mg dose of non-micronized solabegron (Table 10).

TABLE 10
Pharmacokinetics after dosing of 100 mg (calculated) micronized
solabegron and 125 mg micronized solabegron

| | Cₘₐₓ | |
|---|---|---|
| Dose (micronized API) | (ng/mL) | AUC₄₈ (ng · hr/mL) |
| 100 mg (calculated) | 2,040 | 17,000 |
| 125 mg | 2,764 | 22,239 |

1. A pharmaceutical composition consisting of about 100 mg to about 125 mg of micronized solabegron or a pharmaceutically acceptable salt thereof, and a pharmaceutically acceptable excipient, wherein at least 90% of the micronized solabegron has a particle size of about 0.1 micron to 30 microns and wherein the pharmaceutical composition is an immediate release composition.

2. The pharmaceutical composition of claim 1, wherein the pharmaceutically acceptable excipient is selected from the group consisting of a filler, a disintegrant, a binder, a wetting agent, a lubricant, and a glidant, or combinations thereof.

3. The pharmaceutical composition of claim 1, wherein the excipient is selected from the group consisting of mannitol, poloxamer 188, methyl cellulose, croscarmellose sodium, magnesium stearate, colloidal silicon dioxide, and polyvinyl alcohol, or combinations thereof.

4. The pharmaceutical composition of claim 1, wherein the excipient is selected from the group consisting of sucrose, wheat starch, microcrystalline cellulose, talc, lactose monohydrate, calcium carbonate, titanium dioxide, stearic acid, croscarmellose sodium, povidone, polyethylene glycol 8000, colloidal silicon dioxide, ferric oxide, carboxymethylcellulose sodium, white wax, magnesium stearate, and carnauba wax or combinations thereof.

5. The pharmaceutical composition of claim 1, wherein the excipient is selected from the group consisting of colloidal anhydrous silica, calcium hydrogen phosphate dihydrate, cellulose microcrystalline, hypromellose, magnesium stearate, sodium starch glycolate (pH 3.0 to 5.0), stearic acid, and titanium dioxide or combinations thereof.

6. (canceled)

7. The pharmaceutical composition of claim 1, wherein the composition is a tablet, a capsule, a granule, or a powder.

8. (canceled)

9. A method of treating overactive bladder or one or more symptoms thereof, in a subject in need thereof, comprising orally administering to the subject a pharmaceutical composition consisting of a therapeutically effective amount of about 100 mg to about 125 mg of micronized solabegron or a pharmaceutically acceptable salt thereof, and a pharmaceutically acceptable excipient, wherein at least 90% of solabegron particles have a particle size of about 0.1 micron to 30 microns and wherein the pharmaceutical composition is an immediate release composition.

10. The method of claim 9, wherein the pharmaceutical composition is administered twice daily.

11. (canceled)

12. The method of claim 9, wherein the one or more symptoms are selected from increased frequency of urinary urgency, nocturia, increase in urinary micturition frequency, and urinary incontinence, and combination thereof.

13. The method of claim 9, further comprising administering to the subject one or more additional therapeutic agents selected from the group consisting of alpha adrenoceptor blockers, botulinum toxin, purinergics, cannabinoids, transient receptor potential (TRP) protein inhibitors, prostaglandins, percutaneous tibial nerve stimulators, sacral nerve stimulators, 5-alpha reductase inhibitors, phosphodiesterase-5 inhibitors, beta-3 adrenoreceptor agonists, and combination thereof.

14.-17. (canceled)

18. The method of claim 9, wherein the pharmaceutical composition achieves a target AUC₄₈ of about 17,000 ng·hr/mL to about 23,000 ng·hr/mL.

19. The method of claim 18, wherein the therapeutically effective amount of micronized solabegron is about 100 mg.

20. The method of claim 19, wherein the pharmaceutical composition is administered twice a day.

21.-23. (canceled)

24. The pharmaceutical composition of claim 1, wherein the micronized solabegron has a particle size of about 0.1 micron to 10 microns.

25. The method of claim 18, wherein the therapeutically effective amount of micronized solabegron is about 125 mg.

26. The method of claim 25, wherein the pharmaceutical composition is administered twice a day.
